(19) United States
(12) Patent Application Publication
(10) Pub. No.: US 2019/0307860 A1
(43) Pub. Date: Oct. 10, 2019

(54) LATIGLUTENASE (ALV003) FOR USE IN THE TREATMENT OF SYMPTOMATIC CELIAC DISEASE, GLUTEN INTOLERANCE OR GLUTEN SENSITIVITY

(71) Applicant: Immunogenics LLC, Newport Beach, CA (US)

(72) Inventors: Jack A. Syage, Newport Beach, CA (US); Matthew A. Dickason, Newport Beach, CA (US); Jennifer A. Sealey Voyksner, Newport Beach, CA (US)

(21) Appl. No.: 16/463,047

(22) PCT Filed: Nov. 20, 2017
(86) PCT No.: PCT/US17/62602

§ 371 Date: May 22, 2019

Related U.S. Application Data
(60) Provisional application No. 62/426,119, filed on Nov. 23, 2016.

(51) Int. Cl. A61K 38/48; A61P 1/14

(52) U.S. Cl. CPC A61K 38/482; A61P 1/14; C12Y 304/22; C12Y 304/21026; A61K 38/4873

(57) ABSTRACT

A method is provided for reducing the severity and/or frequency of symptoms or clinical manifestations resulting from gluten ingestion by administering to a seropositive patient with symptomatic celiac disease, gluten intolerance, or gluten sensitivity latiglutenase (ALV003) in an amount from about 100 mg to about 1200 mg per day.

### CROSS REFERENCE

This application is a 371 application and claims the benefit of PCT Application No. PCT/US2017/062602, filed Nov. 20, 2017, which claims benefit of U.S. Provisional Patent Application No. 62/426,119, filed Nov. 23, 2016, which applications are incorporated herein by reference in their entirety.

### FIELD OF THE INVENTION

The invention concerns methods for the treatment of symptomatic celiac disease, gluten intolerance or gluten sensitivity. The invention specifically concerns reduction of the severity and/or frequency of symptoms associated with or clinical manifestations of symptomatic celiac disease, gluten intolerance, or gluten sensitivity.

### BACKGROUND OF THE INVENTION

### Celiac Disease

Celiac disease is an acquired chronic immune disorder that develops in susceptible individuals (many of whom are of HLA genotype DQ2 or DQ8) related to an environmental factor, gluten, which is the storage protein of wheat and related grains like rye and barley [(Fasano and Catassi 2001), (Van Heel and West 2006), (Rostom, Murray et al. 2006), (Green and Cellier 2007); a list of the full citation of references cited herein by author name(s) and year of publication is provided at the end of the detailed description of the invention]. The prevalence of celiac disease in Europe and in the United States has been estimated to be approximately 1-2% of the population [(Mäki, Mustalahti et al. 2003), (Fasano, Berti et al. 2003), (West 2003), (Bingley, Williams et al. 2004), (Shan, Qiao et al. 2005), (Van Heel and West 2006), (Green 2007)]. Celiac disease has a wide range of clinical manifestations including latent or silent celiac disease, disease with only mild gastrointestinal disturbances, chronic gastrointestinal symptoms, malabsorption, and/or weight loss. Celiac disease is often diagnosed in patients with isolated iron deficiency anemia.

The ingestion of gluten-containing cereals can also induce manifestations outside the gut (Maki and Collin 1997), such as osteoporosis, peripheral and central nervous system involvement (Hadjivassiliou 2006), mild or severe liver disease, infertility problems, and the classical example is the gluten-induced skin disease, dermatitis herpetiformis. Dermatitis herpetiformis (DH) is a cutaneous manifestation of celiac disease in which an intensely pruritic, herptiform rash can present on the elbows, knees, buttocks, and scalp of a celiac disease patient in response to ingestion of gluten. The rash is characterized by high IgA deposits seen histologically in the upper papillary dermis. The symptoms and histology of the rash improve with adherence to a gluten free diet. Approximately 10% of patients diagnosed with celiac disease will manifest DH. The gluten-induced small bowel pathology in celiac disease is characterized by an inflammatory reaction that is accompanied by villus atrophy and hypertrophy of crypts (Kagnoff 2007).

The only accepted gold standard for celiac disease diagnosis is the finding of gluten-induced small intestinal mucosal injury [(Walker-Smith 1990), (Rostom, Murray et al. 2006)]. Clinical findings are usually equivocal: newly diagnosed patients eating normal gluten-containing food may be totally symptomless or have only vague gastrointestinal symptoms whereas in others symptoms may be severe; in people with extra-intestinal manifestations gastrointestinal symptoms may also be absent, thus having a clinically silent celiac disease. One feature that is common to all however is the manifest gluten-sensitive small intestinal mucosal lesion. In untreated celiac disease, the length of functionally impaired bowel determines the degree of malabsorption and the presence of symptoms does not relate at all to the histological features of the proximal biopsy [(Macdonald, Brandborg et al. 1964), (Marsh and Crowe 1995)]. This also explains why oral glucose tolerance tests, fecal fat excretion, d-xylose excretion tests, hematologic investigations, and radiologic examination of the small bowel fail to distinguish patients with suspected malabsorption from those with or without mucosal atrophy and, thus, frequently give misleading results (Sanderson 1975). Only patients with extensive and severe enteropathy will have evidence of steatorrhea and increased intestinal permeability; in patients with mild-to-moderate enteropathy these tests may remain normal, and therefore these tests are no longer important tools in cases of suspected celiac disease or while monitoring dietary treatment (Farrell and Kelly 2002). Furthermore, recent guidelines and management models for celiac disease diagnosis and treatment in the USA no longer recommend these functional studies [(Hill I D 2005), (Rostom, Murray et al. 2006)]. Instead, during the last two decades, highly sensitive and specific gluten-dependent serum autoantibody tests have been used for celiac disease case finding, population-based screening studies, monitoring the gluten-free diet, and measurement of mucosal relapse on gluten challenge [(Mäki, Hallstrom et al. 1984), (Mäki, Landeaho et al. 1989), (Mäki 1991), (Mäki 1995), (Dieterich 1998), (Sulkanen, Halttunen et al. 1998), (Mustalahti 2002), (Kaukinen, Halme et al. 2002), (Mäki, Mustalahti et al. 2003), (Korponay-Szabo, Raivio et al. 2005), (Collin 2005), (Hill I D 2005), (Holm, Mäki et al. 2006), (Raivio 2006), (Rostom, Murray et al. 2006), (Kurppa 2009)].

For patients with celiac disease, lifelong complete gluten exclusion needs to be strictly followed to avoid a substantially enhanced risk for the development of further complications, such as bone disorders, infertility, and cancer [(Peters, Askling et al. 2003), (Van Heel and West 2006), (Rostom, Murray et al. 2006), (Green and Cellier 2007)]. The mortality rate in patients with celiac disease exceeds that of the general population; however, there is a trend towards reduction in mortality after 1-5 years on a gluten-free diet [(Corrao, Corazza et al. 2001); Rubio-Tapia, et al. 2010)].

Following a completely gluten-free diet is, however, very challenging. Even highly motivated patients who try to maintain a strict dietary regimen are affected due to inadvertent or background exposure to gluten (FDA 2006). As many as 80% of patients with celiac disease who are in clinical remission and who claim to be following a gluten-free diet, have persistent abnormalities in small bowel biopsy specimens [(Lee and Newman 2003), (Bardella, Velio et al. 2007)]. Inadvertent exposure to gluten has been identified as the leading cause of non-responsive celiac disease among clinically diagnosed patients who were presumed to be on a gluten-free diet (Abdulkarim, Burgart et al. 2002). It is evident that a gluten-free diet is more expensive than a so-called ‘normal’ diet; also social life and travel contribute to dietary lapses. Taken together, there is an acute need for non-dietary therapies for celiac disease (Khosla, Gray et al. 2005).

As reviewed by Marsh and Crowe (Marsh and Crowe 1995), time-course studies of gluten challenges provide clear evidence of an inflammatory process, a dose-dependent accumulation of lymphocytes to the epithelium during the lower-dose challenges. Upon further challenge, crypt hyperplasia occurs and lastly, villus effacement is seen (flat mucosal lesion). As evidenced in clinical practice (silent celiac disease), also upon gluten challenge, the mucosal deterioration is often seen before clinical symptoms occur (Maki, Landeaho et al. 1989). When challenging adolescents and young adults with 10 g of gluten per day, and performing a control small intestinal biopsy at the time of seroconversion of the celiac-type autoantibodies, it became evident that the gut mucosa relapsed in 70% of the patients before clinical symptoms occurred. Thus, gluten-induced damage in the small intestinal mucosa is a prerequisite for symptoms and complications of celiac disease, some of which may occur only years or decades after starting gluten ingestion.

Diagnosed celiac disease patients in Finland have typically had at least two upper gastrointestinal endoscopies with multiple biopsies, first at the initial diagnosis and the other approximately one year later to show gut mucosal healing upon a gluten-free diet (Management Model of Celiac Disease in Finland). The Celiac Disease Study Group in Tampere, Finland, has used small intestinal mucosal morphometric analyses to determine ingested wheat-, rye-, and barley- (gluten)-induced mucosal inflammation and mucosal architectural changes in early developing celiac disease, in active and clinically silent disease, during treatment follow-up of patients when the mucosa is healing [(Mäki 1991), (Holm, Mäki et al. 1992), (Holm 1993), (Iltanen, Holm et al. 1999) (Kaukinen, Collin et al. 1998), (Kaukinen, Collin et al. 1999), (Kaukinen 2000), (Kaukinen 2001), (Jarvinen, Kaukinen et al. 2003), (Peraaho, Kaukinen et al. 2003), (Jarvinen 2004), (Collin, Thorell et al. 2004), (Kaukinen 2005), (Salmi 2006)], as well as during gluten challenge (Holm, Mäki et al. 2006). These parameters have included determination of the villus height/crypt depth ratio to establish manifest gluten-induced mucosal architectural change, and counting of the intraepithelial densities of all T lymphocytes (CD3-positive IELs), and densities of αβ+ and γδ+ T cell receptor-bearing IELs to reveal gluten-induced inflammatory changes.

People eating an average Western diet ingest approximately 15-25 g gluten per day. In celiac disease, the onset of symptoms and signs of gluten intolerance may occur in childhood but become evident most often only in adulthood or in the elderly after decades of gluten ingestion. It has been shown in previous clinical gluten challenge studies that older children, adolescents and young adults with long-term treated celiac disease can tolerate well the ingestion of 10-20 g gluten per day [(Mäki, Landeaho et al. 1989), (Holm, Mäki et al. 2006)]. Also, a gluten challenge with repeated small intestinal mucosal biopsies has until fairly recently been mandatory to establish the definite diagnosis of celiac disease, especially in children (in some parts of the world this regimen is still followed). The effect of small gluten loads on the mucosal integrity and a safe gluten threshold in treated celiac disease is still under discussion [(Peraaho, Kaukinen et al. 2003), (Collin 2004) (Catassi, Fabiani et al. 2007), (Hischenhuber 2005)]. One and a half grams to 2 g of daily gluten exposure corresponds to the ingestion of approximately one-half to two-thirds a slice of wheat flour-based bread per day. A drug, to be clinically effective, should be able to significantly reduce or prevent the mucosal deterioration caused by a daily gluten challenge.

### Proteases for the Treatment of Celiac Disease

A promising new approach to treating celiac disease involves the oral administration of proteases, called glutenases, which can degrade gluten. See PCT Pat. Pub. No. 2003/068170; 2005/107786; 2007/044906; 2007/047303; 2008/115411; and 2010/021752; and U.S. Pat. Nos. 7,303,871; 7,320,788; 7,628,985; 7,910,541; and 7,943,312, each of which is expressly incorporated herein by reference.

Cysteine endoprotease (EP) B2 (also known as EPB2), a barley derived protease, and other similar proteases derived from the germinating seeds of the gluten-containing cereals have been identified as effective agents for the detoxification of gluten, the causative agent in celiac disease (including Celiac sprue and dermatitis herpetiformis; see U.S. Pat. No. 7,303,871, incorporated herein by reference). A modified, recombinant form of the barley-derived EPB2 zymogen called “ALV001” (the active form of this enzyme is termed “ALV001* herein) has been used as part of a combination enzyme therapy (including a prolyl endopeptidase (PEP), such as Sphingomonas capsulata PEP) for oral administration to celiac disease patients to aid in the digestion of gluten before it can exert its toxic effects in these patients (see U.S. Pat. No. 7,320,788; U.S. Pat. App. Pub. No. 20080193436; PCT Patent Pub. Nos. 2008/115428; 2008/115411; 2010/021752; and 2010/042203, each of which is expressly incorporated herein by reference). The ALV001 zymogen becomes active (converts to ALV001*) below pH 5, but is not activated at a higher pH.

ALV003 is an especially promising new drug in clinical development that is a mixture of two glutenases. See PCT Pat. Pub. Nos. 2005/107786; 2008/115428; 2008/115411; 2010/021752; and 2010/042203, each of which is expressly incorporated herein by reference. Oral glutenases such as ALV003 help to proteolyze the immunoreactive gluten peptides present in food before they can trigger an immune response in the intestinal mucosa [(Cerf-Bensussan, Matysiak-Budnik et al. 2007), (Piper, Gray et al. 2004), (Gass, Vora et al. 2006), (Pyle, Paaso et al. 2005), (Sollid and Khosla 2005), (Stepniak, Spaenij-Dekking et al. 2006)]. There remains a need for new methods and pharmaceutical compositions that can be used to protect celiac disease patients and other individuals suffering from gluten intolerance from the harmful effects of inadvertent exposure to gluten and to make gluten ingestion safer for them. The present invention meets these needs.

### SUMMARY OF THE INVENTION

In one aspect, the invention concerns a method for reducing the severity and/or frequency of symptoms or clinical manifestations resulting from gluten ingestion comprising administering to a seropositive patient (defined as being positive in either of anti-TG2 IgA, DGP IgA, or DGP Ig) with symptomatic celiac disease, gluten intolerance, or gluten sensitivity latiglutenase (ALV003) in an amount from about 100 mg to about 1200 mg per day.

In one embodiment, the patient has symptomatic celiac disease.

In another embodiment, the patient is moderately to severely symptomatic.

In a third embodiment, the serology status of the patient is determined prior to ALV003 administration.

In other embodiments, determination of the serology status comprises an antibody test selected from the group consisting of anti-gliadin antibodies (AGA), anti-reticulin antibodies (ARA), IgA anti-human tissue transglutaminase (TTG) antibodies (TG2), IgA anti-endomysial antibodies (EMA), and anti-deamidated gliadin peptide (DPG) tests.

In a further embodiment, the patient is seropositive.

In a still further embodiment, the patient remains seropositive despite adhering to a gluten-free diet.

In various embodiments, the symptoms may be self-reported.

In another embodiment, the patient has experienced moderately to severe symptoms of celiac disease within one month from first administration.

In a specific embodiment, the symptoms of gluten ingestion are selected from the group consisting of abdominal pain, bloating, constipation and tiredness, and the treatment reduces the severity and/or the frequency of at least one symptom selected from the group consisting of abdominal pain, bloating, constipation and tiredness.

In one embodiment, the symptom is abdominal pain.

In a second embodiment, the symptom is bloating.

In a third embodiment, the symptom is constipation

In a fourth embodiment, the symptom is tiredness or fatigue.

In another embodiment, the daily dose administered is 1200 mg of ALV003.

In another embodiment, the daily dose administered is 900 mg of ALV003.

In a further embodiment, the daily dose administered is 600 mg of ALV003.

In various embodiments administration occurs at mealtime.

The dose may, for example, be administered with a major meal, such as with major meals three times per day.

For example, the daily amount may be administered in one or more doses taken with food.

In one embodiment, the dose is administered at least once a day for at least a month.

In another embodiment, the dose is administered at least 300 days per year for at least two years.

In yet another embodiment, each dose comprises a dose of ALV001 in powdered form and a dose of ALV002 in powdered form, and the powders are dissolved in a potable liquid to be ingested by the patient.

In a further embodiments, the dose is administered with food containing at least 20 mg but not more than 20 g of gluten, or with food containing no more than about 1 g of gluten, or with food containing no more than about 5 g of gluten, or with food containing no more than about 10 g of gluten.

In another aspect, the invention concerns an article of manufacture comprising a container with latiglutenase (ALV300) therein, and a package insert, wherein the package insert provides instructions to administer latiglutenase (ALV300) for reducing the severity and/or frequency of symptoms or clinical manifestations resulting from gluten ingestion in a seropositive patient with symptomatic celiac disease, gluten intolerance, or gluten sensitivity.

In one embodiment, the symptoms of gluten ingestion are selected from the group consisting of abdominal pain, bloating, constipation and tiredness.

In a further aspect, the invention concerns the use of latiglutenase (ALV003) in the preparation of a medicament for reducing the severity and/or frequency of symptoms or clinical manifestations resulting from gluten ingestion in a seropositive patient with symptomatic celiac disease, gluten intolerance, or gluten sensitivity.

In a still further embodiment, the invention concerns a medicament comprising latiglutenase (ALV003) for use in reducing the severity and/or frequency of symptoms or clinical manifestations resulting from gluten ingestion in a seropositive patient with symptomatic celiac disease, gluten intolerance, or gluten sensitivity.

In all aspects, the symptoms and clinical manifestations resulting from gluten ingestion preferably are, or include, abdominal pain, bloating, constipation and/or tiredness.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1B Celiac Disease Symptom Diary (CDSD)

FIG. 2A-2B Impact of Celiac Disease Symptoms Questionnaire (ICDSQ)

FIG. 3 Patient Global Impressions-Symptoms (PGI-S) & Impact (PGI-I) Questionnaire

FIG. 4A-4C Short Form-12v2 (SF-12v2) Health Survey Questionnaire.

FIG. 5A-5C show histogram plots for baseline and change from baseline for abdominal pain severity for PBO and all latiglutenase doses for week 12. The ordinate is the average total severity per patient for the week.

FIG. 6A-6D: Dose dependent plots for abdominal pain and bloating severity and frequency at week 12. Reduction in symptoms is given by Eq. (1).

FIG. 7A-7B: Bar chart showing reduction in symptom by Eq. (1) for four symptoms for seropositive and seronegative patients for the composite of 600 mg and 900 mg doses. P-values are given.

FIG. 8A-8B: Plots of daily symptom data for a ALV900 patient and for a placebo patient.

FIG. 9: Plots of reduction in symptoms relative to placebo (Eq. (1)) for abdominal pain and bloating severity as a function of baseline severity.

FIG. 10A-10B: Responder analysis defined as percentage of patients who experienced a ≥50% improvement in the severity and frequency of their symptoms relative to placebo.

FIG. 11A-11B: Plots of percent improvement relative to placebo for ICDSQ, PGI, and SF 12v2 criteria for 900 mg dose patients at Week 6 and Week 12 for seropositive and seronegative patients.

FIG. 12A-12F: Plots of the difference in improvement for seropositive vs. seronegative for the data in FIG. 11 and also for 600 mg dose.

### DETAILED DESCRIPTION OF THE INVENTION

### I. Definitions

The term “ALV-1” or “ALV001” is used herein to refer to a zymogenic proenzyme form of cysteine endoprotease B, isoform 1 (EP-B2), naturally occurring in barley. The term, as used herein, specifically includes the polypeptide of SEQ ID NO: 1, with or without the highlighted, vector-derived N- and/or C-terminal residues and with and without the His tags incorporated in the N- and/or C-terminal sequences. The definition further includes post-translational modifications of the proenzyme. In the Examples, “ALV001” is used to refer to the recombinant form of the proenzyme.

The term “ALV-1*” or “ALV001*” is used herein to refer to an active form of the proenzyme ALV-1, as hereinabove defined. The term, as used herein, specifically includes the polypeptide of SEQ ID NO: 2, with or without the highlighted vector-derived C-terminal residues and with or without the C-terminal His tags. In the Examples, “ALV001*” is used to refer to the recombinant form of the active enzyme, which is a modified recombinant version of cysteine endoprotease B, isoform 2 from barley (Hordeum vulgare).

The term “ALV-2” or “ALV002” is used herein to refer to a recombinant version of a prolyl endopeptidase from the bacterium Sphingomonas capsulata (SC-PEP). The term, as used herein, expressly includes the 741 amino acid commercial form of ALV002 (SEQ ID NO:3), with or without the six contiguous histidine residues (hexa-His tag) added in the N-terminal region, and with or without the 38 N-terminal amino acids removed during proteolytic processing.

The terms “ALV-3”, “ALV003”, “IMGX-003” and “latiglutenase” are used interchangeably, and refer to a combination and/or co-administration of ALV-1 and ALV-2 or ALV-1* and ALV-2 or (ALV-1 and ALV-1*) and ALV-2 in a 1:1 (w/w) ratio. In one embodiment, ALV-1 and ALV-2 or ALV-1* and ALV-2 or (ALV-1 and ALV-1*) and ALV-2 are present in the same formulation/dosage form in 1:1 (w/w) ratio (and the formulation/dosage form may include either a formulation in which the two enzymes are admixed or otherwise combined in a single unit dosage form or a formulation in which the two enzymes are in separate dosage form for co-administration). Unless expressly indicated otherwise, the term “ALV-3”, “ALV003”, “IMGX-003” and “latiglutenase” includes combinations comprising ALV-1 and/or ALV-1*. In the Examples, “ALV003”, “IMGX-003” and “latiglutenase” are used to refer to a combination or co-administration of the recombinant forms of ALV001 and/or ALV001* and ALV002.

The term “ALV001/ALV001*” is used herein to mean that either ALV001 or ALV001* can be used.

The terms “celiac sprue” and “celiac disease” are used interchangeably and refer to an autoimmune disease of the small intestine caused by the ingestion of gluten proteins from widely prevalent food sources such as wheat. The terms, as used herein, specifically include clinically silent celiac disease, characterized by absence of gastrointestinal symptoms, and moderate to severe symptomatic celiac disease, characterized by gastrointestinal symptoms that can range from mild to severe. “Celiac disease” as used herein also includes dermatitis herpetiformis.

The term “deleterious effect of gluten ingestion” is used herein to refer to any and all undesired effects of gluten ingestion in a subject, including, without limitation, symptoms and deleterious effects resulting from T lymphocyte-driven immune response in the intestine of celiac disease patients, including gastrointestinal symptoms, such as gluten-induced small intestinal mucosal inflammation and symptoms. The term “deleterious effect of gluten ingestion” also includes any undesired effects of gluten ingestion on the skin of a subject, including, without limitation, symptoms characteristic of dermatitis herpetiformis. The term “deleterious effect of gluten ingestion” specifically includes all symptoms associated with and clinical manifestations of celiac disease, gluten intolerance, or gluten sensitivity, as disclosed herein.

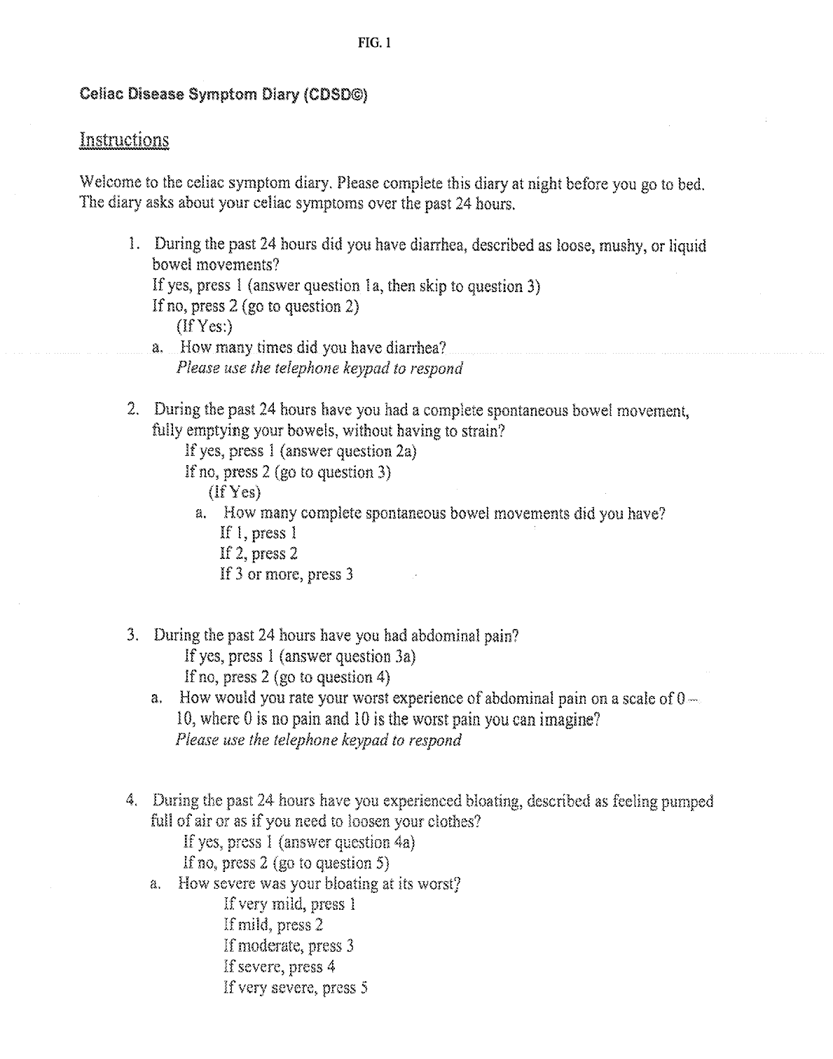

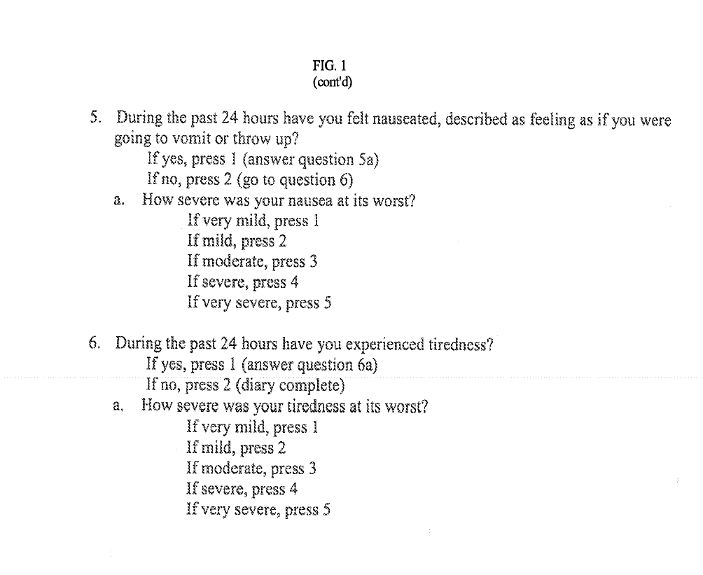

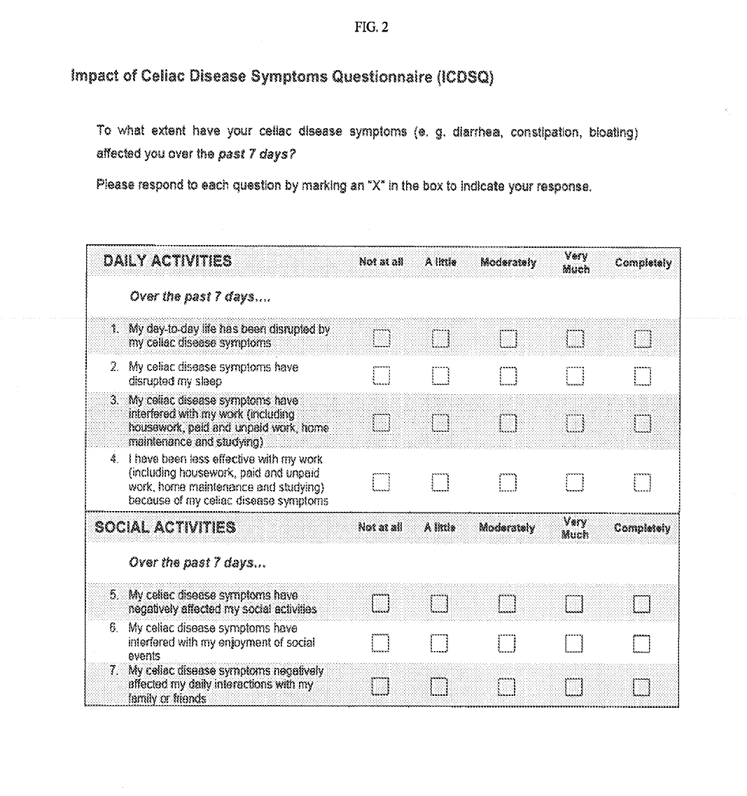

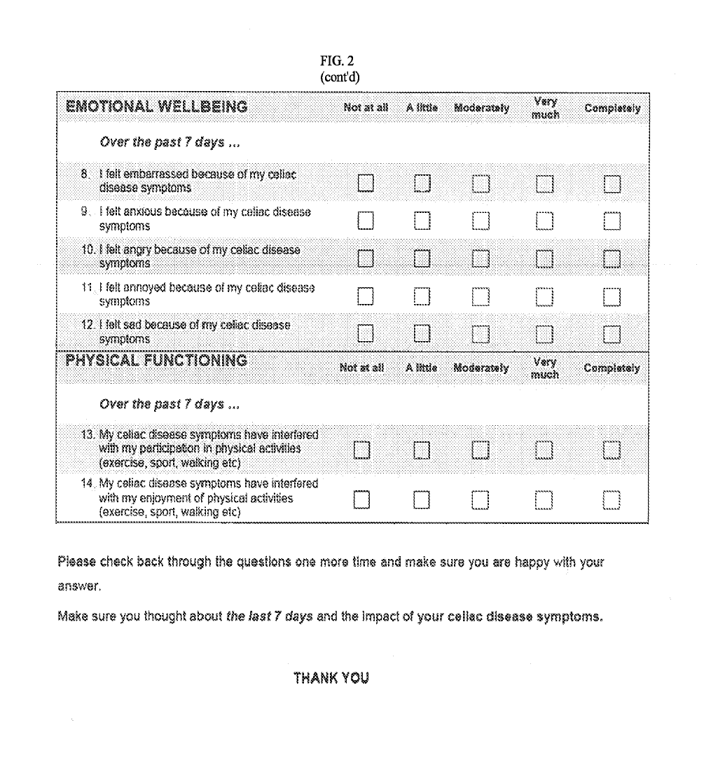

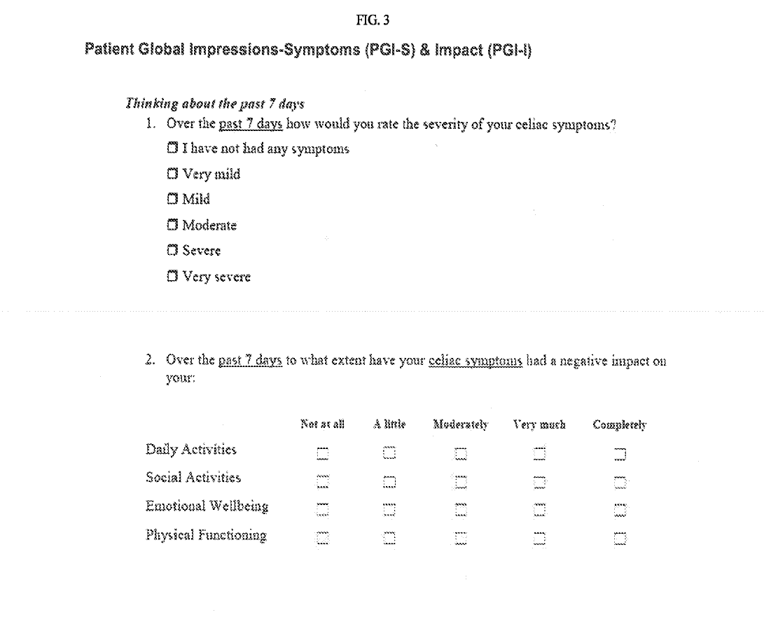

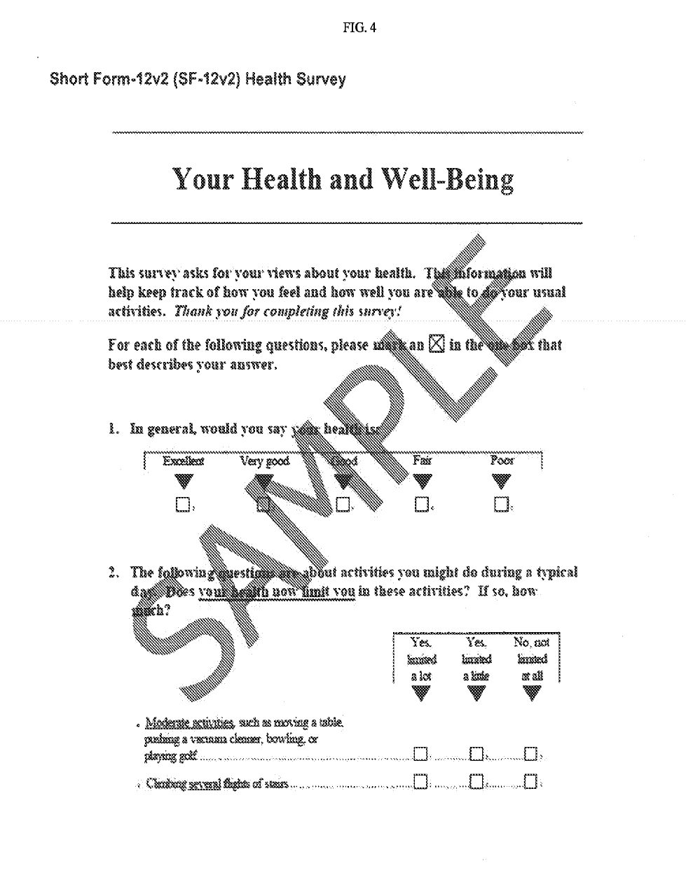

A patient reported outcome (PRO) instrument as used herein, refers to a questionnaire, diary, or any other form of media that can be used to obtain verbal or recorded, e.g. written, typed or tactile, PRO data from a subject. A PRO instrument can be designed to include one or more questions that have been vetted to optimize the form (e.g., graphical; textual or a combination thereof), phrasing or timing of the question to a subject in order to acquire PRO data that is more likely to be valid as compared to data acquired from a non-vetted question. A PRO instrument can be encoded as a computer-executable instruction to be performed on a PRO device. The PRO instrument specifically includes, without limitation, the Celiac Disease Symptom Diary (CDSD) shown in FIG. 1, the Impact of Celiac Disease Symptoms Questionnaire (ICDSQ) shown in FIG. 2, the Patient Global Impressions-Symptoms (PGI-S) & Impact (PGI-I) questionnaire shown in FIG. 3, and the Short Form-12v2 (SF-12v2) Health Survey shown in FIG. 4.

A “clinical trial” as used herein, refers to an experimental trial or test on one or more subjects designed to determine the safety, efficacy, or basis of a label claim for a medical product. A clinical trial includes administering a medical product or a placebo to one or more subjects.

A “clinical endpoint” as used herein, refers to occurrence of, or change in, a disease, a condition, a syndrome, a symptom, a sign or a laboratory measurement in a subject that constitutes one of the target outcomes of a clinical trial.

A “surrogate endpoint” as used herein, is a measure of an effect of a medical product in a clinical trial on a human or non-human subject that correlates with a real clinical endpoint. The surrogate endpoint can be the presence, absence or change in the level of a biomarker.

A “clinical trial endpoint” as used herein, includes both a clinical endpoint and a surrogate endpoint.

A “computer-executable instruction” as used herein, refers to an instruction or a set of instructions able to operate a computer processor to achieve a desired functional result. The desired functional result can be simple, such as the storage of a value in memory, or complex, such as an invocation of an advanced programming interface (API) call that produces sophisticated functionality. The instruction set can be any suitable processor-executable instruction set, including, without limitation, a native machine architecture language, machine language, Java, JavaScript, BASIC, Visual BASIC, C, C++, C#, FORTRAN, Perl, and the like.

The term “PRO device” is used herein in the broadest sense and refers to a device which is used to administer a PRO instrument to a subject to acquire PRO data, including. A PRO device can be, without limitation, a voice recorder, a fax machine, a portable or a fixed electronic device such as a desktop computer or work station terminal.

### II. Detailed Description

ALV003 Mechanism of Action

Gluten has a high proline and glutamine content. This makes it resistant to proteolysis by gastric, pancreatic, and intestinal brush border endo- and exoproteases, which have poor specificity for peptide bonds adjacent to proline and glutamine residues (See (Hausch, Shan et al. 2002), (Shan, Molberg et al. 2002), (Piper, Gray et al. 2004)). As a consequence of the incomplete gastrointestinal proteolysis of gluten, long oligopeptides (such as the 33-mer and 26-mer peptide fragments) accumulate in the small intestine of mammals following ingestion of gluten. Following deamidation by tissue transglutaminase in the intestine, these peptides stimulate an immune response in the intestine of celiac disease patients resulting in structural changes to the lining of the small intestine (Kagnoff 2007). Following the seminal work by Khosla et al. described in PCT Pat. Pub. No. 2003/068170, a number of scientific journal publications have reported the potential for proline- and glutamine-specific endoproteases, referred to as glutenases, as therapeutic agents for celiac disease because of their ability to digest these proteolytically resistant gluten epitopes [(Marti, Molberg et al. 2005), (Shan, Qiao et al. 2005), (Bethune, Strop et al. 2006), (Gass, Vora et al. 2006), (Siegel, Bethune et al. 2006), (Cerf-Bensussan, Matysiak-Budnik et al. 2007), (Gass, Bethune et al. 2007)].

ALV003 is a mixture of two glutenases. The two glutenases that are comprised in ALV003 demonstrate complementary substrate sequence and chain length specificity. If ALV003 comprises the proenzyme form of EPB2, ALV001, upon activation in an acidic environment (as in the stomach) to form ALV001*, proteolyzes gluten at specific glutamine residues and reduces the amount of peptides that are immunostimulatory to T cells derived from celiac disease patients [(Siegel, Bethune et al. 2006), (Bethune, Strop et al. 2006)]. Although ALV002 alone has relatively weak activity on intact gluten proteins, it proteolyzes the peptidic products of ALV001 digestion by cleaving at proline residues [(Shan, Marti et al. 2004), (Gass, Bethune et al. 2007)]. By virtue of their complementary sequence specificity and chain length tolerance for peptides, together ALV001/ALV001* and ALV002 degrade gluten more rapidly and thoroughly than either individual enzyme alone (Gass, Bethune et al. 2007).

The complementary substrate sequence and chain length specificity described above have been demonstrated in vitro and in vivo. ALV003 proteolyzes various forms of gluten (purified gliadin, uncooked gluten flour, and whole wheat bread gluten) in vitro and eliminates >90% of immunoreactive epitopes present. In addition, ALV003 proteolyzed both gluten flour and wheat bread in the stomach of a rat in an in vivo model of gluten digestion. As noted above, at pH values typical of a postprandial stomach (3.5-5), ALV001 activates to its mature form ALV001*, which is active and stable over this pH range. ALV002 contributes to gluten digestion above pH 4. Therefore, ALV003 is active in the stomach during and following a meal. In addition, ALV003 is rapidly proteolyzed by pepsin in both simulated and fasting human gastric fluid (pH 1.8) and also by pancreatin at near neutral pH, providing a mechanism for ALV003 clearance. When incubated in human gastric samples obtained from subjects who had ingested soy milk ex vivo, ALV003 degraded gluten immunoreactive epitopes measured within 30 minutes in a dose-dependent fashion. In vitro, concentrations of ALV003 from 0.25-2.0 mg/mL were able to eliminate >90% of immunoreactive gluten peptides from 0.5-12 mg/mL gluten within 60 min.

Notable favorable properties of ALV003 include its high specificity for gluten and suitability for convenient oral dosing.

Serological Testing to Diagnose Celiac Disease

Celiac disease is characterized by the presence of diverse antibodies in the serum that are made against gliadin, a component of gluten, and connective tissue components, such as tissue transglutaminase antibodies and endomysial antibodies. Tests targeting these antibodies are useful in the diagnosis of celiac disease. In certain embodiments, determination of the serology status of a patient comprises one or more antibody tests selected from the group consisting of anti-gliadin antibodies (AGA), anti-reticulin antibodies (ARA), IgA anti-human tissue transglutaminase (TTG) antibodies (TG2), IgA anti-endomysial antibodies (EMA), and anti-deamidated gliadin peptide (DPG) tests. If at least one of the antibody tests are positive, the patient is considered seropositive.

Pharmaceutical Compositions and Methods of Treatment

Pharmaceutical compositions comprising ALV003, including various formulations for oral administration are known and are described, for example, in WO 2010/021752 and US 2014/0248251-A1, the entire disclosures of which are hereby expressly incorporated by reference. See also, WO 2003/068170; WO 2005/107786; and WO 2008/115411, the disclosures of which are incorporated herein by reference.

In one embodiment, the ALV003 pharmaceutical composition comprises ALV-001, ALV-002, mannitol, potassium citrate, sodium citrate, citric acid, sucrose, TRIS-HCl, EDTA, cysteine hydrochloride, sodium metabisulfite, monthioglycerol, sodium chloride, sucralose, and natural and artificial flavorings.

According to the present invention, ALV003 and pharmaceutical compositions comprising ALV003 can be used in methods for the severity and/or frequency of symptoms associated with or clinical manifestations of celiac disease, gluten intolerance, or gluten sensitivity.

Clinical manifestations of celiac disease, gluten intolerance, or gluten sensitivity include, without limitation, mild gastrointestinal disturbances, chronic gastrointestinal symptoms, malabsorption, weight loss, isolated iron deficiency anemia, various manifestations outside the gut, such as osteoporosis, peripheral and central nervous system involvement, mild or severe liver disease, infertility problems, and the-gluten-induced skin disease, dermatitis herpetiformis. The gluten-induced small bowel pathology in celiac disease is characterized by an inflammatory reaction that is accompanied by villus atrophy and hypertrophy of crypts.

Symptoms of celiac disease, gluten intolerance, or gluten sensitivity include, without limitation, diarrhea, constipation, flatulence, abdominal pain, bloating, nausea, fatigue, tiredness, skin rashes, difficulty thinking, and headache.

Symptoms may range from mild through moderate to severe. The severity of symptoms and/or clinical manifestations can be assessed by patient reported outcome (PRO) instruments, such as, for example, those described in US 2015/0223747 A1, the entire disclosure of which is hereby expressly incorporated by reference. In brief, this PRO instrument may be in the form of a daily or weekly questionnaire that prompts the subject to provide a rating of the extent to which the subject's symptoms have affected the subject's daily life, social activities, emotional wellbeing, and physical functioning. In one embodiment, the PRO instrument also prompts the subject to provide the rating in the form of one of five possible rates, from “not at all” to “completely”, with optional rates of “a little”, “moderately”, and “very much” (or equivalents thereto). In various embodiments, the PRO instrument may be provided in the form of a bi-weekly or monthly questionnaire or simply to provide a point in time evaluation (no specific time period specified). This PRO instrument, called a “Celiac Disease Symptom Diary”, which may be referred to herein as an “CDSD”, has a variety of applications, including but not limited to: to measure symptoms in a clinical trial of a drug or other therapy to treat celiac disease, gluten intolerance, or gluten sensitivity, including but not limited to measuring symptoms in connection with a clinical trial endpoint; to measure symptoms to determine if a subject should be treated for celiac disease, gluten intolerance, or gluten sensitivity; to monitor the status of a patient's condition over time; to identify subjects that should be tested for celiac disease; to monitor the efficacy of a treatment; to determine if a subject should be treated, including but not limited to providing an economic rationale for such treatment, for celiac disease, gluten intolerance, or gluten sensitivity; and to aid a physician in determining or monitoring the health status of a patient that may have celiac disease, gluten intolerance, or gluten sensitivity.

While the PRO instrument, called an “Celiac Disease Symptom Diary” (CDSD) is preferred for measuring the severity and frequency of symptoms, other PRO instruments are also available and can be used, including, for example, PROs that have focused on patient's health-related quality of life.

The PRO instruments used herein specifically include the Celiac Disease Symptom Diary (CDSD), Impact of Celiac Disease Symptoms Questionnaire (ICDSQ), the Patient Global Impressions-Symptoms (PGI-S) & Impact (PGI-I) Questionnaire, and the Short Form 12-v2 Health Survey (SF-12v2) Questionnaire described in the Example (FIGS. 1-4).

The severity of the disease can also be determined using medical diagnostic methods known in the art, such as upper gastrointestinal endoscopies, biopsies, small intestinal mucosal morphometric analyses, determination of the villus height/crypt depth ratio to establish manifest gluten-induced mucosal architectural change, and measuring the intraepithelial densities of all CD3+(T) lymphocytes and densities of αβ+ and γδ+ T cell receptor-bearing IELs to reveal gluten-induced inflammatory changes.

While thrice daily (TID) administration is contemplated in various embodiments of the invention, QD administration may also be practiced, i.e., when a patient is consuming only one gluten-containing (or potentially gluten-containing) meal per day. Thus, ALV003 may be administered when a patient is ingesting food suspected of containing, or known to contain, gluten.

Optionally, the patient's serology status may be determined prior to administration of the compositions herein. Determination of the serology status may comprise an antibody test, such as anti-gliadin antibodies (AGA), anti-reticulin antibodies (ARA), IgA anti-human tissue transglutaminase (TTG) antibodies (TG2), and IgA anti-endomysial antibodies (EMA), and anti-deamidated gliadin peptide (DGP) tests.

Administration may occur at mealtime, such as with a major meal or meals, e.g. one to three times, such as three times, per day.

A typical daily dose for oral administration of ALV003 is in the range of about 100 mg to about 3 g, such as, for example, 100 mg, 300 mg, 450 mg, 600 mg, 900 mg, or 1200 mg. As discussed earlier, the daily dose can be reached by one or more administrations, typically taken with food.

People eating an average Western diet ingest approximately 15-25 g gluten per day; a typical slice of whole-wheat bread contains approximately 4-5 g of gluten. In various embodiments, ALV003 is administered with food containing at least about 15 mg, or at least about 20 mg, but not more than about 25 g of gluten, or no more than about 1 g of gluten, or no more than about 2 g of gluten, or no more than about 3 g of gluten, or no more than about 5 g of gluten, or no more than about 10 g of gluten.

In another embodiment, ALV003 has equal amounts of (ALV001 and/or ALV001*), and ALV002, by weight or by units of activity, including embodiments wherein ALV001/ALV-001* has a specific activity of at least 5000 or more proteolytic activity units per mg, and said ALV002 has a specific activity of at least 3000 or more proteolytic activity units per mg.

Further details of the invention will be illustrated by the following non-limiting examples.

All references cited throughout the disclosure are hereby expressly incorporated by reference in their entirety.

### Example

ALV003-1221, a Phase 2b, Randomized, Double-Blind, Placebo-Controlled Dose-Ranging Study of the Efficacy and Safety of ALV003 Treatment in Symptomatic Celiac Disease Patients Maintained on a Gluten-Free Diet

Celiac disease (CD) is an autoimmune disease triggered by the ingestion of gluten that often causes severe gastrointestinal symptoms for those afflicted with the disease. At present, there is no available treatment other than a gluten-free diet (GFD), which is very difficult to achieve and many patients continue to experience significant symptoms.

The present ALV003-1221 clinical trial (NCT01917630) investigated the effectiveness of latiglutenase (ALV-003), an orally administered mixture of two recombinant gluten-specific digestive enzymes, for reducing major symptoms resulting from inadvertent gluten ingestion. In particular, ALV003-1221 was a double-blind, placebo-controlled, dose-ranging clinical study that assessed the efficacy and safety of latiglutenase (ALV003) in symptomatic, established, celiac disease (CD) patients who reported following a GFD for at least one year prior to randomization. Patients with documented moderate or severe symptoms and villous atrophy, villus height:crypt depth (Vh:Cd)≤2.0 (typically), were randomized to placebo or active treatment for 12 or 24 weeks and then underwent follow-up duodenal biopsy. Symptoms were recorded daily throughout the study.

### Patients and Methods

Eligibility Criteria

Ages Eligible for Study: 18 Years to 80 Years

Genders Eligible for Study: Both

Inclusion Criteria:
Ages 18 to 80 years
Physician diagnosed celiac disease
Adherence to a gluten-free diet
Experiencing symptoms of celiac disease over a 1-month period
Willing to take study medication for 12 weeks
Willing to comply with all study procedures
Sign informed consent

Exclusion Criteria:
Active inflammatory bowel disease
Active dermatitis herpetiformis
Use of certain specific medications prior to entry
History of alcohol or illicit drug abuse in previous 6 months
Pregnant or lactating
Received any experimental drug within 30 days of enrollment
Uncontrolled chronic disease or condition

Study Design

Patients were randomized to one of six treatment groups: Placebo (PBO), ALV003 100 mg (A100), ALV003 300 mg (A300), ALV003 450 mg (A450), ALV003 600 mg (A600), and ALV003 900 mg (A900). Patients were allocated by dose level approximately in a 3:1:2:1:2:1 ratio. Table I shows the distribution of completed patients who were seronegative and seropositive for each treatment group. Seropositive status was determined by any one of the following tests exceeding a threshold value; anti-TG2 IgA, DGP IgA, DGP IgG.

Completed Patient Sample Size

TABLE I

| | Number of Patients | | |
|---|---|---|---|
| Treatment | Seropos | Seroneg | All serology |
| PBO | 54 | 68 | 122 |
| ALV100 | 20 | 27 | 47 |
| ALV300 | 35 | 40 | 75 |
| ALV450 | 15 | 23 | 38 |
| ALV600 | 35 | 45 | 80 |
| ALV900 | 14 | 22 | 36 |
| Total | 173 | 225 | 398 |

### Statistical Analysis

A statistical analysis was performed assessing objectives related to the quality of life of patient-reported outcomes. The objectives of this analysis were as follows:
To determine the effect of different dose levels of ALV003 at 12 and 24 weeks on celiac disease symptom frequency and severity as measured by the Celiac Disease Symptoms Questionnaire (CDSD) (FIG. 1).
To assess the effect of different dose levels of ALV003 on quality of life at 12 and 24 weeks as measured by the Impact of Celiac Disease Symptoms Questionnaire (ICDSQ) (FIG. 2) and the Short Form 12-v2 Health Survey (SF-12v2) (FIG. 4).
To explore potential responder definitions for the CDSD.
To explore the feasibility of a composite endpoint.

Patient-Reported Outcome Instruments and Scoring

Celiac Disease Symptom Diary (CDSD)

The CDSD is a daily diary administered across a seven-day period that assesses common celiac symptoms (diarrhea, constipation, abdominal pain, bloating, nausea, and tiredness). The presence or absence of each of the symptoms over the previous 24 hours is reported. If the respondent indicates the presence of a particular symptom, contingent follow-up questions are asked to assess the symptom severity for that particular symptom. See FIG. 1 for the CDSD.

For each daily diary entry, the presence of each symptom will be indicated as the following responses to the questions: diarrhea (Q1=1), constipation (Q2=1; i.e., the absence of a constipation), abdominal pain (Q3=1), bloating (Q4=1), nausea (Q5=1), tiredness (Q6=1).

For each symptom, within the given study week (7 days), a weekly frequency score will be calculated by summing the number of times during the week each symptom was indicated. Thus, for each symptom, the weekly frequency score will range from 0 (never indicating the symptom during the study week) to 7 (indicating the symptom every day of the study week).

It should be stated explicitly, however, that the clinical definition of constipation generally requires a week of follow up (i.e., less than 3 complete spontaneous bowel movements within a 1-week timeframe). Hence, the weekly frequency score for Q2 will represent the number of days during the week where the patient did not have any complete spontaneous bowel movements, rather than the number of days where the patient was constipated in a strict clinical sense.

An overall symptom frequency score will also be computed that will be equal to the number of days in the week (range: 0 to 7) where any of the gastrointestinal symptoms (Q1, Q2, Q3, Q4, and/or Q5) was indicated. The tiredness symptom (Q6) will not be included in the calculation of the overall symptom frequency score.

Each time the patient indicates a particular symptom in the daily diary, a follow-up question will be asked about severity. For each symptom, with the exception of constipation, a daily severity score will be computed as follows:
Diarrhea Daily Severity=Response to Q1a; (contingent upon Q1=1)
Abdominal Pain Daily Severity=Response to Q3a; (contingent upon Q3=1)
Bloating Daily Severity=[(2.25)*(Response to Q4a)]-1.25; (contingent upon Q4=1)
Nausea Daily Severity=[(2.25)*(Response to Q5a)]-1.25; (contingent upon Q5=1)
Tiredness Daily Severity=[(2.25)*(Response to Q6a)]-1.25; (contingent upon Q6=1)

For each of the above symptoms, if the contingent question criterion is not met, then the daily severity score will be assigned a value of 0. Thus, each daily symptom score will be on a 0 to 10 scale. For each symptom, within the given study week (7 days), a weekly severity score will be calculated by summing the daily severity scores (range: 0 to 10) over the week, which will yield a range of 0 to 70 for each weekly symptom severity score.

For constipation, the weekly severity score will be calculated by pooling together data across the entire study week (rather than calculating daily severity scores then summing to compute a weekly severity score). Based on the responses to Q2a, if the patient indicates 3 or more complete spontaneous bowel movements (CSBMs) taking together all days across the particular study week, then the weekly severity score will be set to 0 (i.e., no constipation). Otherwise, if the patient reports less than 3 complete spontaneous bowel movements taking together all the days across the particular study week, the weekly severity score will be calculated as follows:

Constipation Weekly Severity=[(−31.5)*(Weekly number of CSBMs)]+70;

(contingent upon Weekly number of CSMBs<3)

Hence, the Constipation Weekly Severity score will range from 0 to 70 based on the number of complete spontaneous bowel movements that the patient indicates across the entire study week.

A weekly Overall Severity Score will be calculated by summing the weekly severity scores of the five gastrointestinal symptoms (Diarrhea Severity, Constipation Severity, Abdominal Pain Severity, Bloating Severity, Nausea Severity). Thus, the Overall Severity Score will range from 0 to 350.

Impact of Celiac Disease Symptoms Questionnaire (ICDSQ)

The ICDSQ will be used to assess the impact of patients' celiac symptoms over the previous week at Visit 4 (Day 1), Visit 5 (Week 6), Visit 6 (Week 12), and at Visit 7 (Week 18) and Visit 8 (Week 24) for those patients participating in Study Period 3. The questionnaire has a 7-day recall period and is comprised of 14 items with four domains: Daily Activities (4 items), Social Activities (3 items), Emotional Well-being (5 items) and Physical Functioning (2 items).

Each item has five response options ranging from 0 (“not at all”) to 4 (“completely”). Each domain will be scored by computing the mean of the domain items. An overall ICDSQ score will be calculated by summing the four mean domain scores. See FIG. 2 for the ICDSQ.

Patient Global Impression-Symptoms (PGI-S)

Although the PGI-S will not be analyzed as an outcome, it will be used to define responder categories for the CDSD (as described in the Statistical Methods section below). The PGI-S is a single global assessment item that asks participants to rate the severity of their celiac symptoms over the previous seven days on a 6-point scale (0=“I have not had any symptoms” to 5=“very severe”). See FIG. 3 for the PGI-S and PGI-I items.

Short Form-12v2 (SF-12v2(Health Survey)

The SF-12v2 Health Survey is a shorter version of the SF-36 Health Survey that will ask patients to answer 12 questions that measure physical and mental health at Day 1, Week 6, Week 12, and at Week 18 and Week 24 for those patients participating in Study Period 3. The SF-12v2 covers eight health domains (Physical Functioning, Role Physical, Bodily Pain, General Health, Vitality, Social Functioning, Role Emotional, and Mental Health) which comprise two component summary measures (Physical Component Summary and Mental Component Summary). All scores range from 0 to 100.

The SF-12v2 will be scored using the QualityMetric algorithm. Additionally the SF-6D Utility Index will be calculated. Scoring algorithm details along with a description of the SF-12v2 instrument itself have been previously described (Ware 2002). See FIG. 4 for the SF-12 v2 Health Survey.

All data analyses were generated using SAS software, Version 9.2 of the SAS System for Windows or higher. Copyright ©2003 SAS Institute Inc. See also, Ware J E Jr, Kosinski M, Turner-Bowker D M, Gandek B. How to Score Version 2 of the SF-12 Health Survey (With a Supplement Documenting Version 1). Lincoln, R.I.: QualityMetric Incorporated, 2002.

### Results

CDSD Severity and Frequency Analysis

Dose Dependence:

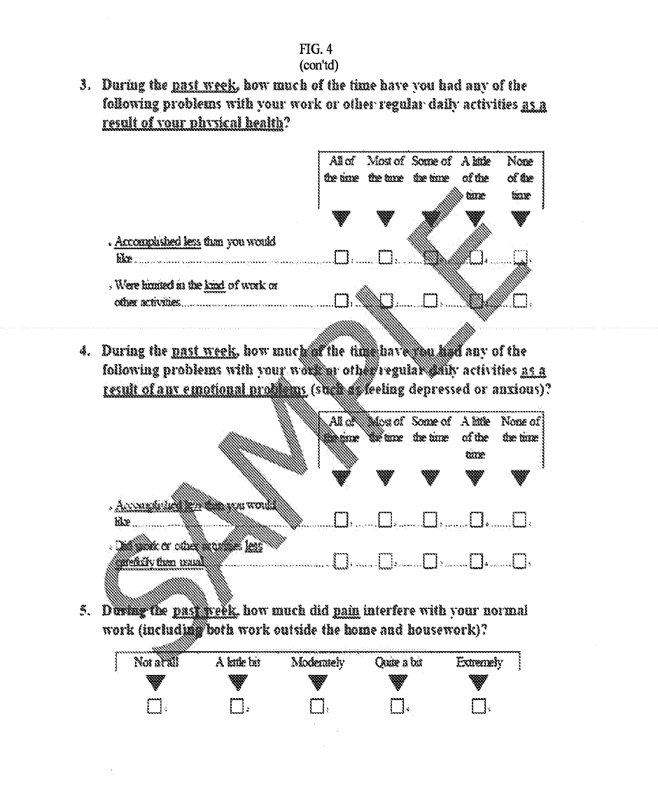

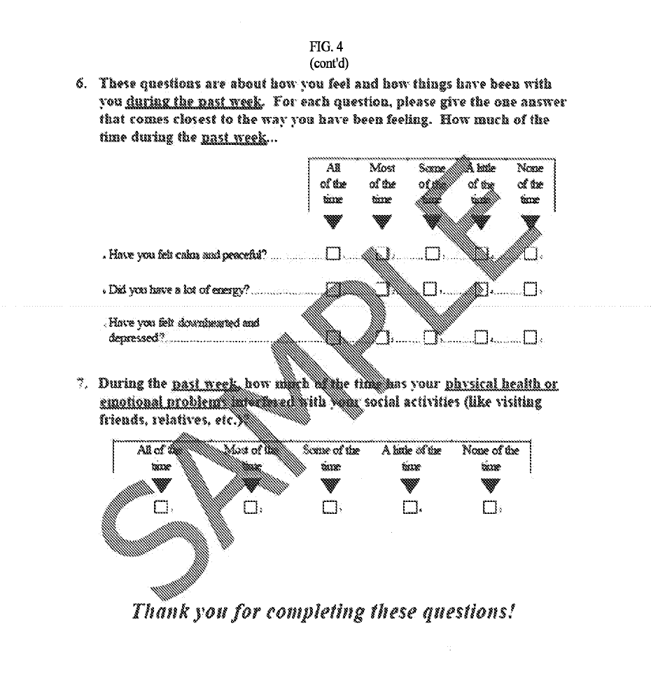

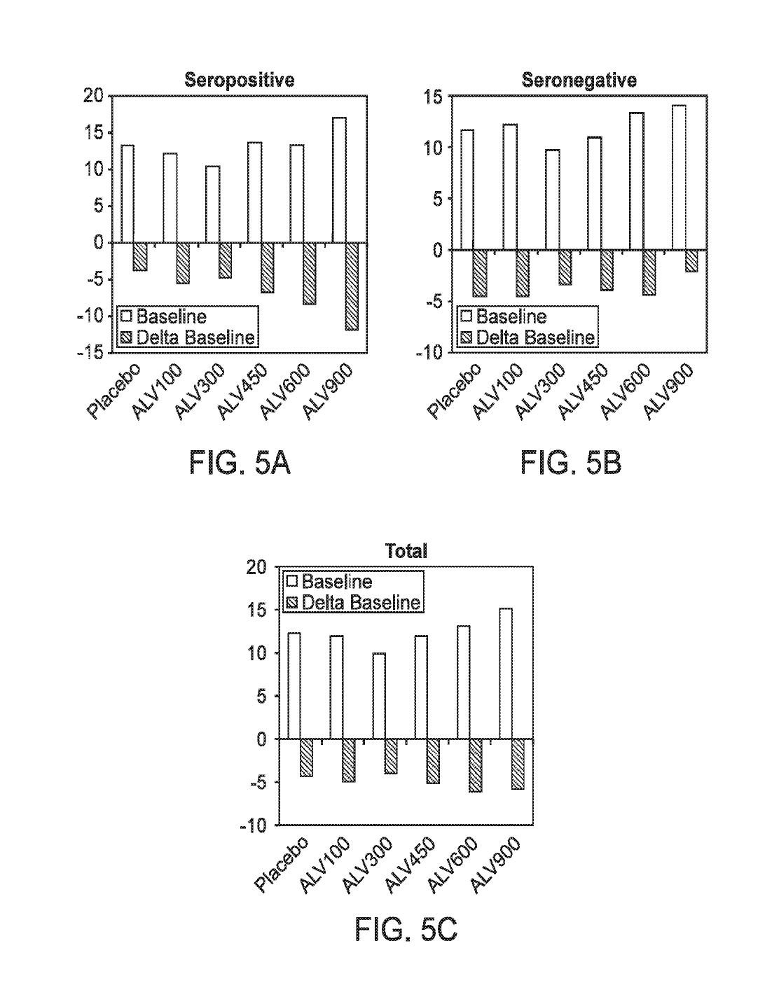

FIG. 5A-C shows baseline and change from baseline for abdominal pain for week 12 stratified by serology. There is a clear trend toward a dose-dependent reduction of symptoms under latiglutenase for seropositive patients. There does not appear to be a drug benefit for seronegative patients. Symptom improvement was evaluated by:

RIS_{dose} (%)=[(ΔB_{dose}/B_{dose})−(ΔB_{PBO}/B_{PBO})]/(1−(ΔB_{PBO}/B_{PBO}) Eq. (1)

where RIS_{dose} (%) is the percent reduction in symptoms, B_{dose} is the baseline value (i.e., the total severity score of the symptom in the week prior to randomization), and ΔB_{dose} is the change in baseline value cumulative for all patients for a particular dose. The subscript PBO represents the placebo dose population. The (1−(ΔB_{PBO}/B_{PBO})) term in the denominator accounted for the improvement in a symptom due to latiglutenase activity relative to the placebo effect; as a result, RIS_{dose} could assume values between 0% (corresponding to the placebo effect) and 100% (full recovery of the symptom).

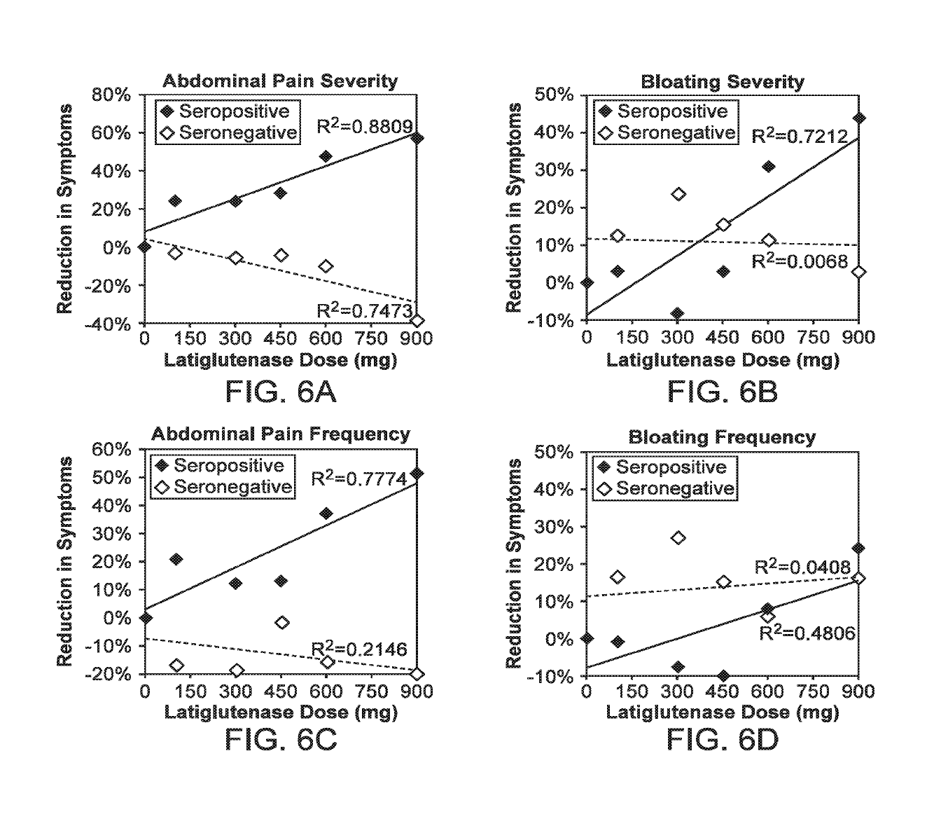

FIG. 6A-D presents dose dependent plots for abdominal pain and bloating severity and frequency according to Eq. (1). It is apparent that there is a distinct dose-dependent symptom improvement response for seropositive, but not seronegative patients. Tiredness and constipation (as measured by complete spontaneous bowel movements (CSBMs) per week) follow similar trends as for abdominal pain and bloating.

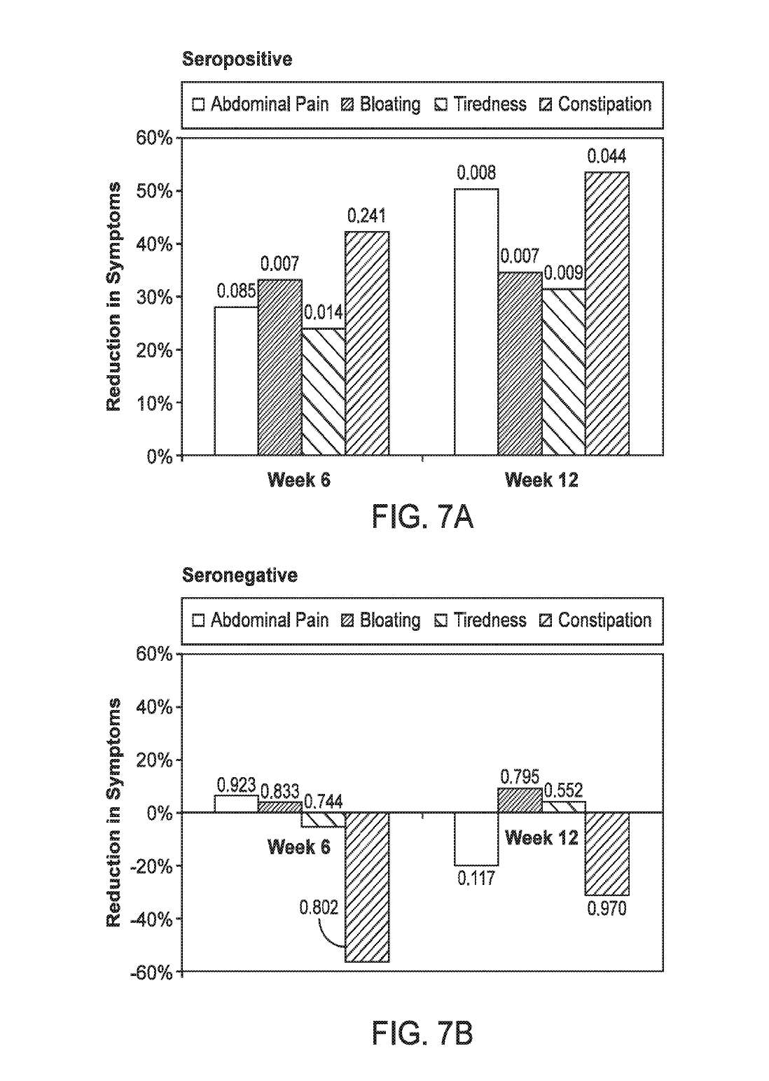

Table II summarizes the percent improvement for the 600 mg and 900 mg doses (ALV600 and ALV900, respectively) relative to PBO for abdominal pain, bloating and tiredness for week 6 and week 12 using the CDSD severity and frequency data. This analysis is from the same data set plotted in FIGS. 6A-D and gives more complete evidence of latiglutenase activity. The p-values were calculated by analysis of covariance (ANCOVA). FIG. 7A, B shows data similar to Table II but for the composite of 600 mg and 900 mg, which improves the statistics as represented by p-values. There are several trends to note:
There is a marked improvement for the higher latiglutenase doses, such as ALV600 and ALV900
The data show distinct dose dependence as also evidenced in FIG. 6
There is an increased benefit for week 12 relative to week 6
Seropositive patients were much more strongly benefitted than seronegative patients
The p-values indicate statistical significance for symptom improvements for almost all categories of symptom domain, dose, and period under treatment

It is also worth noting that improvement as measured by MS is generally greater for severity than for frequency, which is expected based on the definitions for each. Severity represents the average total score for severity, which inherently has frequency in it whereas frequency does not distinguish the severity of each event. These results indicate that latiglutenase reduces both the frequency of events as well as the severity of each event.

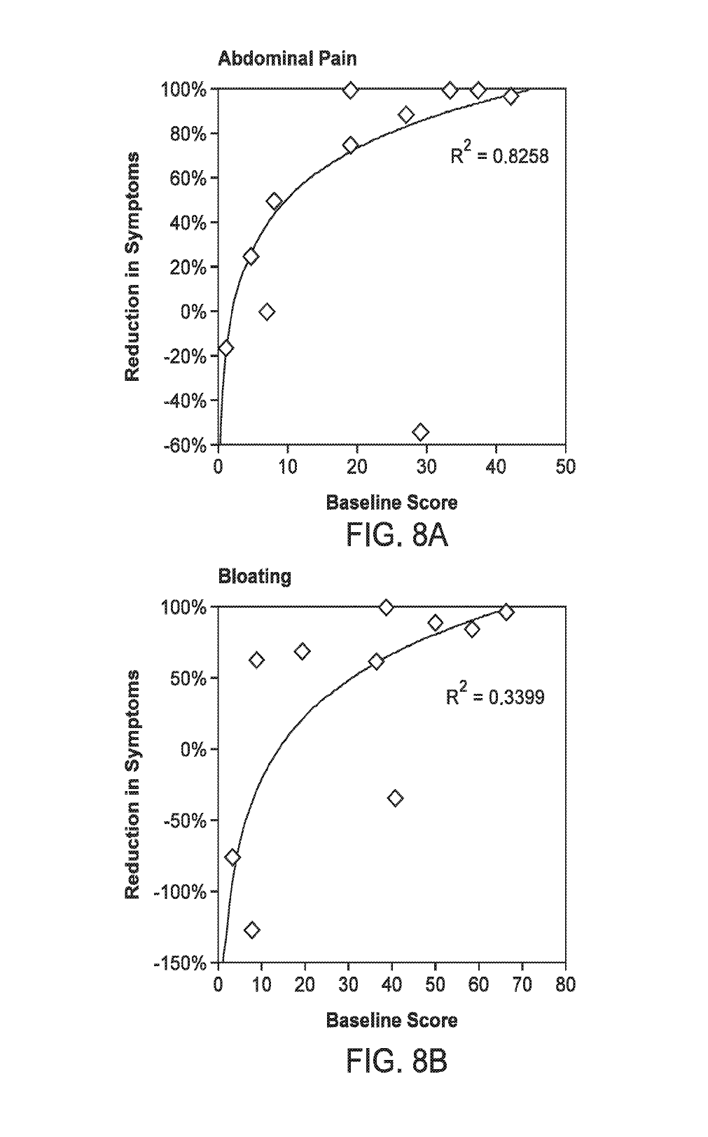

FIG. 8A, B shows the MS as a function of baseline severity for abdominal pain and bloating for the 900 mg treatment group. There is a trend toward greater MS with greater baseline severity indicating that latiglutenase is more effective for patients who are more symptomatic.

TABLE II
Symptom improvements, RIS, and p-values relative to PBO based on CDSD Severity and
Frequency Scales.

| Abdominal Pain | | | | Bloating | | | | Tiredness | | | |
|---|---|---|---|---|---|---|---|---|---|---|---|
| Severity | p-value | Freq. | p-value | Severity | p-value | Freq. | p-value | Severity | p-value | Freq. | p-value |

ALV900

| | | | | | | | | | | | | |
|---|---|---|---|---|---|---|---|---|---|---|---|---|
| Week 6 | | | | | | | | | | | | |
| Seropos | 30.8% | 0.165 | 17.2% | 0.445 | 32.1% | 0.021 | 11.4% | 0.364 | 21.7% | 0.070 | 18.9% | 0.074 |
| Seroneg | 6.8% | | 9.5% | | 7.5% | | 13.6% | | 9.3% | | 8.3% | |
| Total | 17.1% | | 12.5% | | 17.3% | | 12.1% | | 14.4% | | 12.3% | |
| Week 12 | | | | | | | | | | | | |
| Seropos | 47.2% | 0.038 | 41.9% | 0.034 | 32.5% | 0.023 | 18.2% | 0.189 | 17.7% | 0.164 | 8.7% | 0.391 |
| Seroneg | −17.1% | | −6.4% | | 7.3% | | 18.1% | | 13.9% | | 18.0% | |
| Total | 10.1% | | 11.9% | | 16.7% | | 17.6% | | 15.0% | | 14.1% | |

ALV600

| | | | | | | | | | | | | |
|---|---|---|---|---|---|---|---|---|---|---|---|---|
| Week 6 | | | | | | | | | | | | |
| Seropos | 18.2% | 0.165 | 18.3% | 0.141 | 17.9% | 0.040 | 6.9% | 0.317 | 16.3% | 0.028 | 15.2% | 0.038 |
| Seroneg | 6.3% | | −1.3% | | 5.7% | | 5.9% | | −10.9% | | −15.1% | |
| Total | 11.5% | | 7.2% | | 11.1% | | 6.3% | | 1.4% | | −1.3% | |
| Week 12 | | | | | | | | | | | | |
| Seropos | 34.0% | 0.026 | 28.9% | 0.039 | 17.5% | 0.040 | 2.8% | 0.521 | 25.3% | 0.002 | 19.7% | 0.012 |
| Seroneg | −1.4% | | −7.3% | | 13.1% | | 7.1% | | −1.4% | | −7.6% | |
| Total | 14.1% | | 8.2% | | 15.1% | | 5.3% | | 10.8% | | 5.0% | |

A strong trial effect (more than normal conformance to a gluten-free diet during the trial period) was evident from the objective histologic and this is evident in the subjective symptom PRO data as well as evidenced in FIG. 5; although there may be an additional placebo effect that cannot be differentiated from the trial effect.

Finally, it is important to note that for nausea and diarrhea where little improvement due to latiglutenase is observed, that there is no evidence of symptom worsening further bolstering the safety of latiglutenase.

CDSD Daily Symptom Analysis

We analyzed the raw data from the CDSD tool. This entailed reporting of events and severity for the six symptom domains for all patients for all days of the trial including the weeks 1-4 baseline period and the weeks 5-8 randomization period. The study then ran for 12 weeks for all patients and another 12 weeks for about a quarter of the completed patients. In this analysis we defined three periods: weeks −8 to 0 (days 1-56), which we used for baseline calculations since the CDSD continued to be administered during the randomization period preceding the start of the trial, weeks 1-6 (days 57-98) of the trial and weeks 7-12 (days 99-140) of the trial.

Individual Plots:

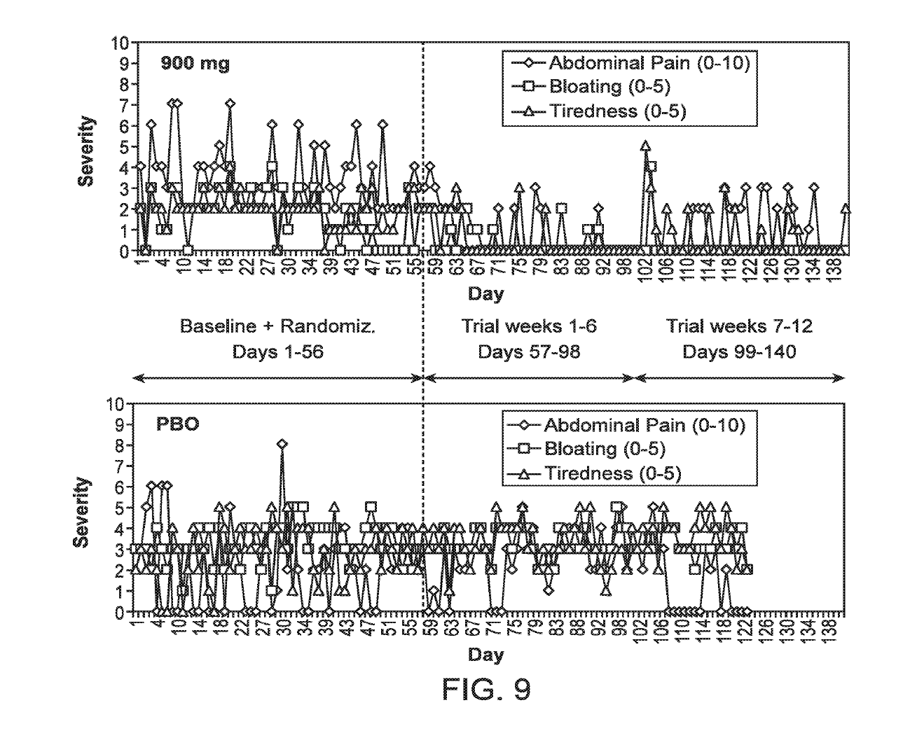

In FIG. 9 are plots of daily symptom responses for two randomly chosen seropositive patients, one on ALV900 and the other on PBO. These two examples are reasonably representative of each trial arm, but are presented for illustration purposes only as they do not consist of the complete statistical data for all patients (Table I). The most noticeable trends are:
The recruited group of patients (e.g., moderate to severe systems) experience frequent symptom episodes of severe intensity as evidenced by the numerous events appearing in the day 1-56 period before the trial.
While some patients experience continuous on-going gastrointestinal related symptoms, there is also clear evidence that the most severe symptom events appear as flares.
For the ALV900 patient there is distinct improvement in the symptom responses for abdominal pain, bloating, and tiredness.
For the PBO patient there is insignificant improvement in these symptoms

Integration Over Time Periods:

We now report on the results of the daily data consolidated for all seropositive patients in the ALV900 and PBO arm. Table III summarizes the frequency of symptom events as a function of severity for abdominal pain, bloating, and tiredness for the ALV900 and PBO seropositive arms. The results are presented for the pre-trial period (days 1-56) and the two halves of the trial period (days 57-98 and 99-140, respectively). The most notable observations are:
The PBO patients on the whole experienced a reduction in the frequency of symptom events, which can be attributed to a combination of a trial effect (more than normal conformance to a gluten-free diet during the trial period) and placebo effect (due to changes in patient's subjective ratings).
The ALV900 patients show a much greater reduction in the frequency of symptom events, overcoming the trial/placebo effect for the PBO arm. Because the ALV900 arm also is expected to have the same level trial/placebo effect as the PBO arm, the increased reduction in symptom events is attributable to the drug activity. A similar, but proportionately lower symptom improvement was observed for ALV600.
The improvement relative to PBO increased for the second half of the trial consistent with the frequency and severity data in Table II.

There appears to be a trend toward greater symptom improvement relative to PBO the greater the severity of the symptom event. This trend was also observed for the Week 12 average data in FIG. 8.

Analysis was also conducted for seronegative patients and there was no significant symptom benefit observed for abdominal pain, bloating, and tiredness. One can also conclude that there was no noticeable worsening in these symptoms for seronegative patients due to the use of latiglutenase, although there is more noise in these data due to small differences between large numbers.

TABLE III
Tabulation of frequency of symptom events as a function of severity. (The
7x events/report is for the highest severity scale and normalizes the
frequency to 7 days overcoming non-compliant reporting days; the close
values to the calculated values indicate that non-compliance is a minor
source of deviation in the data.)

| | | |
|---|---|---|
| Days 1-56 | Days 57-98 | Days 99-140 |

| | | | | | | |
|---|---|---|---|---|---|---|
| Severity | total | per week | total | per week | total | per week |

PBO
Abdominal Pain

| | | | | | | |
|---|---|---|---|---|---|---|
| ≥1 | 1278 | 2.96 | 691 | 2.13 | 454 | 1.40 |
| ≥2 | 1213 | 2.81 | 642 | 1.98 | 427 | 1.32 |
| ≥3 | 969 | 2.24 | 522 | 1.61 | 345 | 1.06 |
| ≥4 | 661 | 1.53 | 333 | 1.03 | 251 | 0.77 |
| ≥5 | 437 | 1.01 | 230 | 0.71 | 179 | 0.55 |
| ≥6 | 294 | 0.68 | 159 | 0.49 | 124 | 0.38 |
| ≥7 | 172 | 0.40 | 94 | 0.29 | 76 | 0.23 |
| ≥8 | 86 | 0.20 | 55 | 0.17 | 47 | 0.15 |
| ≥9 | 30 | 0.07 | 12 | 0.04 | 22 | 0.07 |
| ≥10 | 9 | 0.02 | 4 | 0.01 | 7 | 0.02 |

| | | | |
|---|---|---|---|
| 7 x events/report | 0.02 | 0.01 | 0.03 |

Bloating

| | | | | | | |
|---|---|---|---|---|---|---|
| ≥1 | 1912 | 4.43 | 1085 | 3.35 | 837 | 2.58 |
| ≥2 | 1778 | 4.12 | 1020 | 3.15 | 778 | 2.40 |
| ≥3 | 1342 | 3.11 | 788 | 2.43 | 608 | 1.88 |
| ≥4 | 567 | 1.31 | 368 | 1.14 | 314 | 0.97 |
| ≥5 | 158 | 0.37 | 147 | 0.45 | 98 | 0.30 |

| | | | |
|---|---|---|---|
| 7 x events/report | 0.33 | 0.44 | 0.36 |

Tiredness

| | | | | | | |
|---|---|---|---|---|---|---|
| ≥1 | 2544 | 5.89 | 1085 | 3.35 | 1140 | 3.52 |
| ≥2 | 2399 | 5.55 | 1020 | 3.15 | 1077 | 3.32 |
| ≥3 | 1945 | 4.50 | 788 | 2.43 | 823 | 2.54 |
| ≥4 | 910 | 2.11 | 368 | 1.14 | 397 | 1.23 |
| ≥5 | 241 | 0.56 | 147 | 0.45 | 113 | 0.35 |

| | | | |
|---|---|---|---|
| 7 x events/report | 0.50 | 0.44 | 0.42 |

ALV900
Abdominal Pain

| | | | | | | |
|---|---|---|---|---|---|---|
| ≥1 | 436 | 3.89 | 198 | 2.36 | 120 | 1.43 |
| ≥2 | 414 | 3.70 | 187 | 2.23 | 109 | 1.30 |
| ≥3 | 315 | 2.81 | 137 | 1.63 | 75 | 0.89 |
| ≥4 | 180 | 1.61 | 69 | 0.82 | 34 | 0.40 |
| ≥5 | 102 | 0.91 | 36 | 0.43 | 25 | 0.30 |
| ≥6 | 52 | 0.46 | 18 | 0.21 | 15 | 0.18 |
| ≥7 | 25 | 0.22 | 8 | 0.10 | 8 | 0.10 |
| ≥8 | 7 | 0.06 | 4 | 0.05 | 6 | 0.07 |
| ≥9 | 0 | 0.00 | 3 | 0.04 | 4 | 0.05 |
| ≥10 | 0 | 0.00 | 2 | 0.02 | 0 | 0.00 |

| | | | |
|---|---|---|---|
| 7 x events/report | 0.00 | 0.02 | 0.00 |

Bloating

| | | | | | | |
|---|---|---|---|---|---|---|
| ≥1 | 526 | 4.70 | 313 | 3.73 | 164 | 1.95 |
| ≥2 | 480 | 4.29 | 258 | 3.07 | 133 | 1.58 |
| ≥3 | 332 | 2.96 | 148 | 1.76 | 76 | 0.90 |
| ≥4 | 102 | 0.91 | 47 | 0.56 | 24 | 0.29 |
| ≥5 | 23 | 0.21 | 13 | 0.15 | 2 | 0.02 |

| | | | |
|---|---|---|---|
| 7 x events/report | 0.15 | 0.14 | 0.02 |

Tiredness

| | | | | | | |
|---|---|---|---|---|---|---|
| ≥1 | 740 | 6.61 | 392 | 4.67 | 316 | 3.76 |
| ≥2 | 709 | 6.33 | 375 | 4.46 | 299 | 3.56 |
| ≥3 | 552 | 4.93 | 274 | 3.26 | 190 | 2.26 |
| ≥4 | 237 | 2.12 | 99 | 1.18 | 82 | 0.98 |
| ≥5 | 43 | 0.38 | 36 | 0.43 | 19 | 0.23 |

| | | | |
|---|---|---|---|
| 7 x events/report | 0.29 | 0.37 | 0.23 |

Responder Analysis

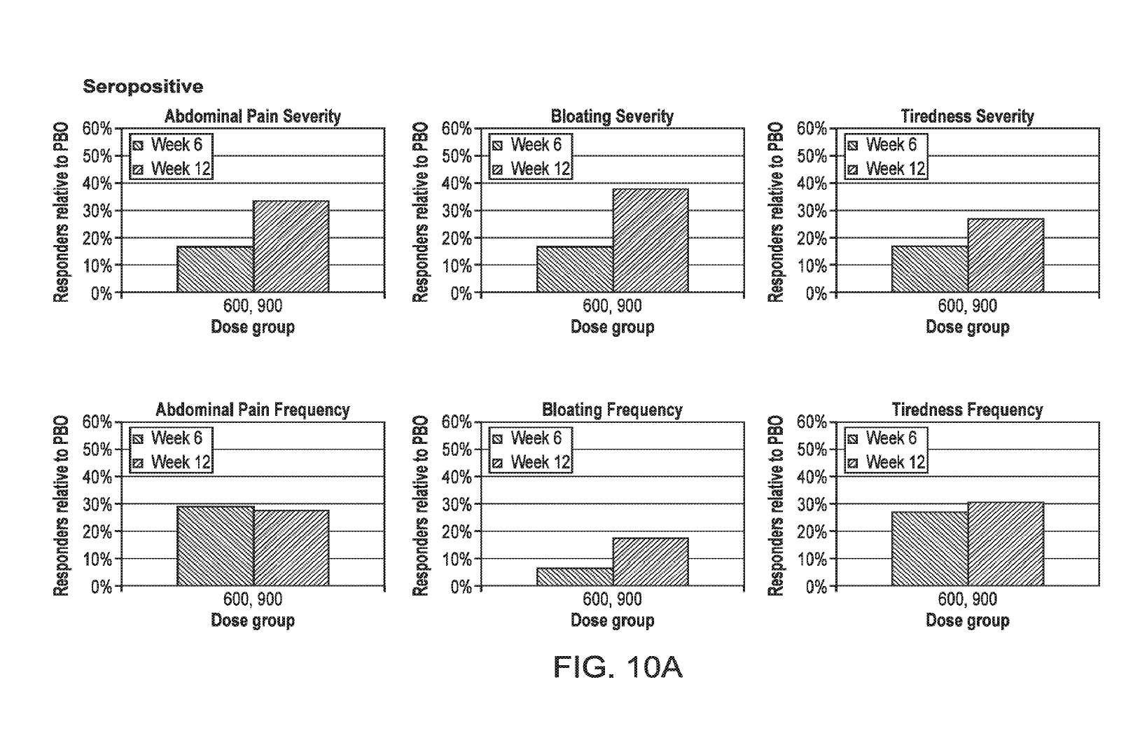

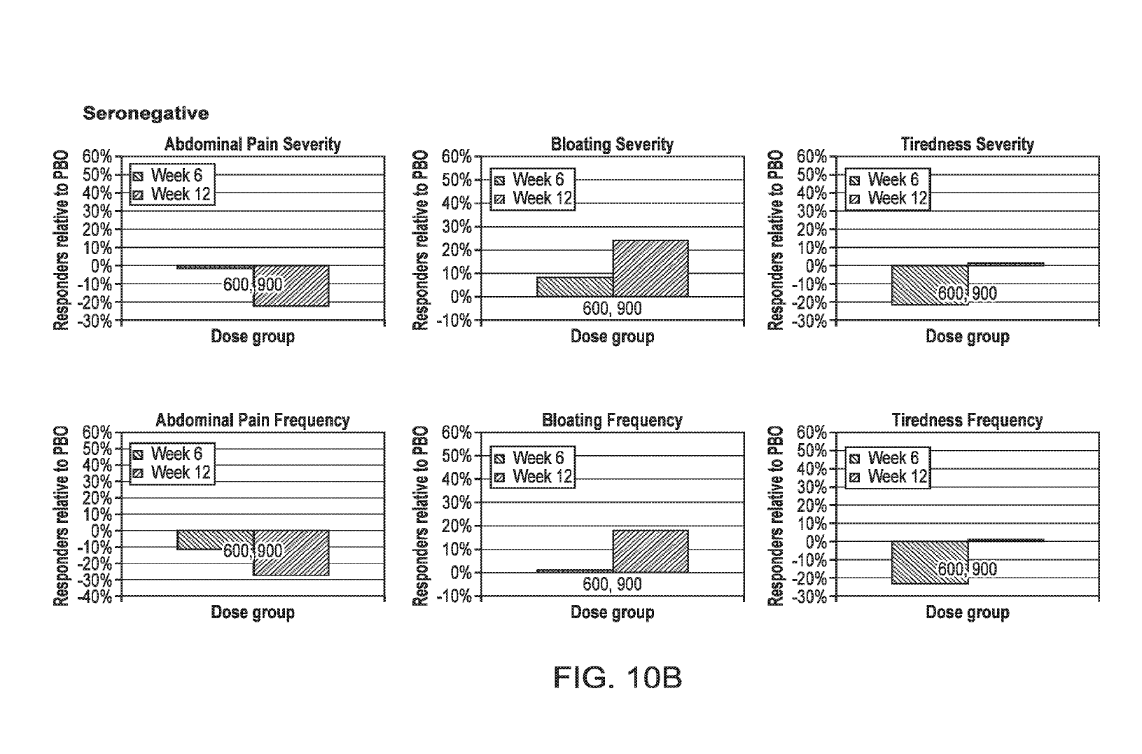

We performed a responder analyses on the symptom data. The definition for responder in this work is the percentage of individuals who exceeded a RIS relative to placebo in a symptom domain based on the severity and frequency scales. For RIS threshold we chose ≥50%, but we also calculated for ≥30%, which gave similar results FIG. 10A, B shows the ≥50% results for severity and frequency for abdominal pain, bloating, and tiredness for weeks 6 and 12 of the study for the composite of 600 mg and 900 mg treatment groups. In all cases and for weeks 6 and 12 a positive responder effect is observed relative to PBO. There is also a general trend toward increasing percentage of responders for increasing doses. The seronegative patient population on average did not experience any apparent benefit from latiglutenase relative to placebo (FIG. 10B).

### Quality of Life

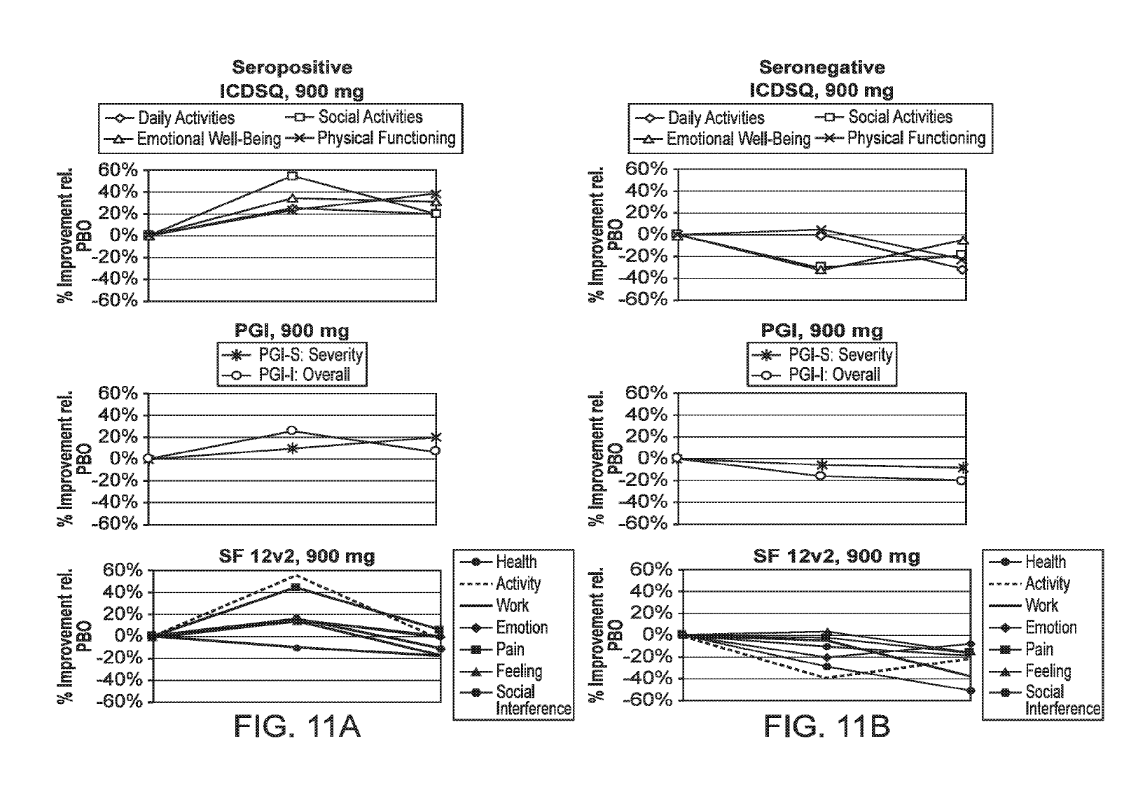

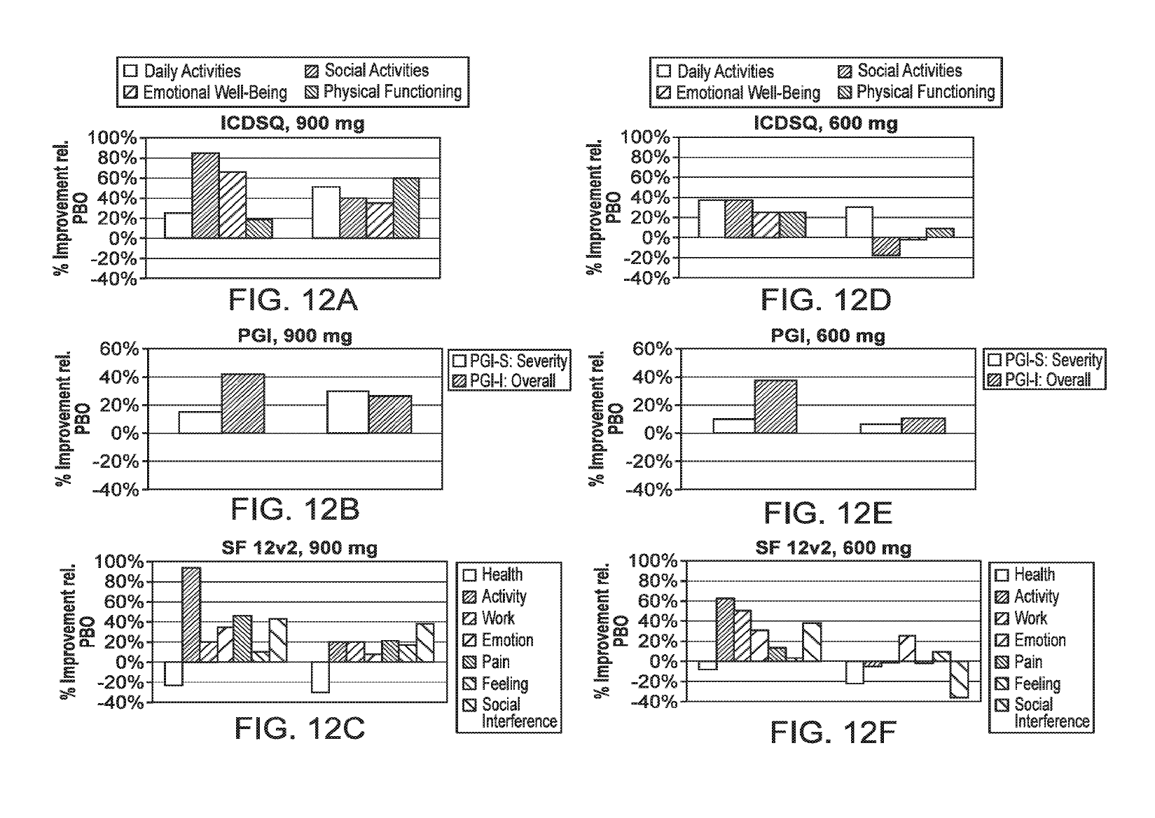

Quality of Life (QOL) was measured with the ICDSQ, PGI-S, PGI-I, and SF-12v2 instruments. FIG. 11A, B shows the improvement for various domains of each of these instruments for seropositive and seronegative patients for the 900 mg treatment groups. There is a clear indication of greater improvement in these domains for the seropositive patients. FIG. 12A-F plots the difference in the improvements between seropositive and seronegative patients for these domains for the 900 mg and 600 mg treatment groups. These data show that the improvement is greater for seropositive patients for nearly all domains. The data also show that the difference in improvement between seropositive and seronegative patients still exists for the 600 mg group, but the difference is not as great as for the 900 mg group, showing a dose dependence similar to that observed for the CDSD results in FIG. 6.

### REFERENCES

Abdulkarim, A. S., L. J. Burgart, et al. (2002). “Etiology of nonresponsive celiac disease: results of a systematic approach.” Am J Gastroenterol 97(8): 2016-21.
Anderson, R. P., P. Degano, et al. (2000). “In vivo antigen challenge in celiac disease identifies a single transglutaminase-modified peptide as the dominant A-gliadin T-cell epitope.” Nat Med 6(3): 337-42.
Anderson, R. P., D. A. van Heel, et al. (2006). “Antagonists and non-toxic variants of the dominant wheat gliadin T cell epitope in coeliac disease.” Gut 55(4): 485-91.
Bardella, M. T., P. Velio, et al. (2007). “Coeliac disease: a histological follow-up study.” Histopathology 50(4): 465-471.
Bethune, M. T., P. Strop, et al. (2006). “Heterologous expression, purification, refolding, and structural-functional characterization of EP-B2, a self-activating barley cysteine endoprotease.” Chem Biol 13(6): 637-47.
Bingley, P. J., A. J. K. Williams, et al. (2004). “Undiagnosed coeliac disease at age seven: population based prospective birth cohort study.” BMJ 328(7435): 322-323.
Catassi, C., E. Fabiani, et al. (2007). “A prospective, double-blind, placebo-controlled trial to establish a safe gluten threshold for patients with celiac disease.” Am J Clin Nutr 85(1): 160-6.
Catassi, C., M. Rossini, et al. (1993). “Dose dependent effects of protracted ingestion of small amounts of gliadin in coeliac disease children: a clinical and jejunal morphometric study.” Gut 34(11): 1515-9.
Cerf-Bensussan, N., T. Matysiak-Budnik, et al. (2007). “Oral proteases: a new approach to managing coeliac disease.” Gut 56(2): 157-60.
Ciclitira, P. (1984). “Clinical testing of gliadin fractions in coeliac patients.” Clin Sci 66(3): 357-64.
Collin, G. (2004). “Complete small intestinal mucosal recovery is obtainable in the treatment of celiac disease.” Gastrointestinal Endoscopy 59(1): 158.
Collin, P. (2005). “Antiendomysial and antihuman recombinant tissue transglutaminase antibodies in the diagnosis of coeliac disease: a biopsy-proven European multicentre study.” Eur J Gastroenterol Hepatol 17(1): 85-91.
Collin, P., L. Thorell, et al. (2004). “The safe threshold for gluten contamination in gluten-free products. Can trace amounts be accepted in the treatment of coeliac disease?” Alimentary Pharmacology & Therapeutics 19(12): 1277-1283.
Corrao, G., G. R. Corazza, et al. (2001). “Mortality in patients with coeliac disease and their relatives: a cohort study.” The Lancet 358(9279): 356-361.
Dieterich, W. (1998). “Autoantibodies to Tissue Transglutaminase as Predictors of Celiac Disease.” Gastroenterology 115: 1317-1321.
Dimenas, E. (1993). “Methodological aspects of evaluation of Quality of Life in upper gastrointestinal diseases.” Scand J Gastroenterol Suppl 199: 18-21.
Farrell, R. J. and C. P. Kelly (2002). “Celiac Sprue.” N Engl J Med 346(3): 180-188.
Fasano, A., I. Berti, et al. (2003). “Prevalence of celiac disease in at-risk and not-at-risk groups in the United States: a large multicenter study.” Arch Intern Med 163(3): 286-92.
Fasano, A. and C. Catassi (2001). “Current approaches to diagnosis and treatment of celiac disease: an evolving spectrum.” Gastroenterology 120(3): 636-51.
FDA (2006). “Guidance for Industry Patient-Reported Outcome Measures: Use in Medical Product Development to Support Labeling Claims.”
FitzSimmons, S. (1997). “High Dose Pancreatic Enzyme supplements and fibrosing colonopathy in children with cystic fibrosis.” Massachusetts Medical Society 336(18): 6.
Gass, J., M. T. Bethune, et al. (2007). “Combination enzyme therapy for gastric digestion of dietary gluten in patients with celiac sprue.” Gastroenterology 133(2): 472-80.
Gass, J., H. Vora, et al. (2006). “Effect of barley endoprotease EP-B2 on gluten digestion in the intact rat.” J Pharmacol Exp Ther 318(3): 1178-86.
Glade, M. J., D. Kendra, et al. (2001). “Improvement in protein utilization in nursing-home patients on tube feeding supplemented with an enzyme product derived from Aspergillus niger and Bromelain.” Nutrition 17(4): 348-350.
Green, P. H. (2007). “Where are all those patients with Celiac disease?” Am J Gastroenterol 102(7): 1461-3.
Green, P. H. and C. Cellier (2007). “Celiac disease.” N Engl J Med 357(17): 1731-43.
Hadjivassiliou, M. (2006). “Autoantibody targeting of brain and intestinal transglutaminase in gluten ataxia.” Department of Neurology and Gastroenterology 66: 373-377.
Hallert, C. (1998). “Quality of Life of Adult Coeliac Patients Treated for 10 Years.” Scand J Gastroenterol.
Hamilton, J. (1972). “Childhood celiac disease: response of treated patients to a small uniform daily dose of wheat gluten.” J Pediatrics(81): 885-93.
Hausch, F., L. Shan, et al. (2002). “Intestinal digestive resistance of immunodominant gliadin peptides.” Am J Physiol Gastrointest Liver Physiol 283(4): G996-G1003.
Hauser, W. (2007). “Development and Validation of the Celiac Disease Questionnaire (CDQ), a Disease-specific Health-related Quality of Life Measure for Adult Patients With Celiac Disease.” J Clin Gastroenterol 41: 157-166.
Hauser, W. (2007). “Predictors of Irritable Bowel-Type Symptoms and Healthcare-Seeking Behavior Among Adults With Celiac Disease.” Psychosomatic Medicine 69: 370-376.
Hegde, V. L. and Y. P. Venkatesh (2004). “Anaphylaxis to excipient mannitol: evidence for an immunoglobulin E-mediated mechanism.” Clinical & Experimental Allergy 34(10): 1602-1609.
Hill I D, D. M., Liptak G S, Colletti R B, Fasano A, Guandalini S, Hoffenberg E J, Horvath K, Murray J A, Pivor M, Seidman E G; (2005). “Guideline for the Diagnosis and Treatment of Celiac Disease in Children: Recommendations of the North American Society for Pediatric Gastroenterology, Hepatology and Nutrition.” North American Society for Pediatric Gastroenterology, Hepatology and Nutrition. 40(1): 1-19.
Hischenhuber, C. (2006). “Allergen management in the food industry—potential and limitations.” Mol Nutr Food Res 49(1): 4-5.
Hogberg, L., L. Stenhammar, et al. (1997). “Anti-endomysium and anti-gliadin antibodies as serological markers for a very late mucosal relapse in a coeliac girl.” Acta Paediatrica 86(3): 335-336.
Holm, K., M. Maki, et al. (1992). “Intraepithelial [gamma][delta] T-cell-receptor lymphocytes and genetic susceptibility to coeliac disease.” The Lancet 339(8808): 1500-1503.
Holm, K., M. Maki, et al. (2006). “Oats in the treatment of childhood coeliac disease: a 2-year controlled trial and a long-term clinical follow-up study.” Alimentary Pharmacology & Therapeutics 23(10): 1463-1472.
Holm, K. H. (1993). “Correlation of HLA-DR alleles to jejunal mucosal morphology in healthy first-degree relatives of coeliac disease patients.” European Journal of Gastroenterology & Hepatology 5(1): 35-40.
Iltanen, Holm, et al. (1999). “Changing jejunal [gamma][delta] T cell receptor (TCR)-bearing intraepithelial lymphocyte density in coeliac disease.” Clinical & Experimental Immunology 117(1): 51-55.
Jarvinen, T. T. (2004). “Villous Tip Intraepithelial Lymphocytes as Markers of Early-Stage Coeliac Disease.” Scand J Gastroenterol 5.
Jarvinen, T. T., K. Kaukinen, et al. (2003). “Intraepithelial lymphocytes in celiac disease.” The American Journal of Gastroenterology 98(6): 1332-1337.
Kagnoff, M. F. (2007). “Celiac disease: pathogenesis of a model immunogenetic disease.” J Clin Invest 117(1): 41-9.
Kane, S. and M. J. Goldberg (2000). “Use of bromelain for mild ulcerative colitis.” Ann Intern Med 132(8): 680.
Kaukinen, K. (2000). “Intolerance to Cereals Is Not Specific for Coeliac Disease.” Scand J Gastroenterol 9.
Kaukinen, K. (2001). “Celiac Disease Without Villous Atrophy Revision of Criteria Called for.” Digestive Diseases and Sciences 46(4): 879-887.
Kaukinen, K. (2005). “Immunohistochemical Features in Antiendomysium Positive Patients with Normal Villous Architecture.” American Journal of Gastroenterology: 675-676.
Kaukinen, K. (2005). “Small-bowel muscosal transglutaminase 2-specific IgA deposits in coeliac disease without villous atrophy: A prospective and randomized clinical study.” Scand J Gastroenterol 40: 564-572.
Kaukinen, K., P. Collin, et al. (1998). “Small-Bowel Mucosal Inflammation in Reticulin or Gliadin Antibody-Positive Patients without Villous Atrophy.” Scandinavian Journal of Gastroenterology 33: 944-949.
Kaukinen, K., P. Collin, et al. (1999). “Wheat Starch-Containing Gluten-Free Flour Products in the Treatment of Coeliac Disease and Dermatitis Herpetiformis: A Long-Term Follow-up Study.” Scandinavian Journal of Gastroenterology 34: 163-169.
Kaukinen, K., L. Halme, et al. (2002). “Celiac disease in patients with severe liver disease: gluten-free diet may reverse hepatic failure.” Gastroenterology 122(4): 881-888.
Khosla, C., G. M. Gray, et al. (2005). “Putative efficacy and dosage of prolyl endopeptidase for digesting and detoxifying gliadin peptides.” Gastroenterology 129(4): 1362-3; author reply 1363.
Korponay-Szabo (2004). “In vivo targeting of intestinal and extraintestinal transglutaminase 2 by coeliac autoantibodies.” Gut 53: 641-48.
Korponay-Szabo, I. R., T. Raivio, et al. (2005). “Coeliac disease case finding and diet monitoring by point-of-care testing.” Aliment Pharmacol Ther 22(8): 729-37.
Kuitunen P, K. I., Savialhti E. (1982). “Morphometric study of the jejunal mucosa in various childhood enteropathies with special reference to intraepithelial lymphocytes.” J Pediatr Gastroenterol Nutr 1: 525-31.
Kurppa, K. (2009). “Diagnosing Mild Enteropathy Celiac Disease: A Randomized, Controlled Clinical Study.” Gastroentérologie Clinique et Biologique 136: 816-823.
Lee, A. and J. M. Newman (2003). “Celiac diet: Its impact on quality of life.” Journal of the American Dietetic Association 103(11): 1533-1535.
Lohiniemi, S. (1998). “Measuring quality of life in coeliac disease patients.” Changing Features of Coeliac Disease. The Finnish Coeliac Society. (Tampere 1998): 73-77.
Lopez, M. and L. Edens (2005). “Effective prevention of chill-haze in beer using an acid proline-specific endoprotease from Aspergillus niger.” J Agric Food Chem 53(20): 7944-9.
Macdonald, W. C., L. L. Brandborg, et al. (1964). “Studies of Celiac Sprue. Iv. The Response of the Whole Length of the Small Bowel to a Gluten-Free Diet.” Gastroenterology 47: 573-89.
Mäki, M. (1995). “The humoral immune system in coeliac disease.” Baillieres Clin Gastroenterol 9(2): 231-49.
Mäki, M. (1991). “Increase in gamma/delta T cell receptor bearing lymphocytes in normal small bowel mucosa in latent coeliac disease.” Gut 32: 1412-4.
Mäki, M. and P. Collin (1997). “Coeliac disease.” The Lancet 349(9067): 1755-1759.
Mäki, M., O. Hallstrom, et al. (1984). “Evaluation of a serum IgA-class reticulin antibody test for the detection of childhood celiac disease.” J Pediatr 105(6): 901-5.
Mäki, M., M. L. Landeaho, et al. (1989). “Postpubertal gluten challenge in coeliac disease.” Arch Dis Child 64: 1604-7.
Mäki, M., K. Mustalahti, et al. (2003). “Prevalence of Celiac Disease among Children in Finland.” N Engl J Med 348(25): 2517-2524.
Marsh, M. N. and P. T. Crowe (1995). “5 Morphology of the mucosal lesion in gluten sensitivity.” Bailliere's Clinical Gastroenterology 9(2): 273-293.
Marti, T., O. Molberg, et al. (2005). “Prolyl endopeptidase-mediated destruction of T cell epitopes in whole gluten: chemical and immunological characterization.” J Pharmacol Exp Ther 312(1): 19-26.
McHorney, C. A., J. E. Ware, Jr., et al. (1994). “The MOS 36-item Short-Form Health Survey (SF-36): III. Tests of data quality, scaling assumptions, and reliability across diverse patient groups.” Med Care 32(1): 40-66.
Mustalahti, K. (2002). “GlutenFree Diet and Quality of Life in Patients with Screen-Detected Celiac Disease.” Eff Clin Pract.
Mustalahti, K., S. Lohiniemi, et al. (2002). “Gluten-free diet and quality of life in patients with screen-detected celiac disease.” Eff Clin Pract 5(3): 105-13.
O'Leary, C. (2004). “Celiac Disease and the Transition from Childhood to Adulthood: A 28-Year Follow-Up.” American Journal of Gastroenterology.
Pasricha, P. J., D. E. Fleischer, et al. (1994). “Endoscopic perforations of the upper digestive tract: a review of their pathogenesis, prevention, and management.” Gastroenterology 106(3): 787-802.
Paterson, B. M., K. M. Lammers, et al. (2007). “The safety, tolerance, pharmacokinetic and pharmacodynamic effects of single doses of AT-1001 in coeliac disease subjects: a proof of concept study.” Aliment Pharmacol Ther 26(5): 757-66.
Peraaho, M., K. Kaukinen, et al. (2003). “Wheat-starch-based gluten-free products in the treatment of newly detected coeliac disease: prospective and randomized study.” Alimentary Pharmacology & Therapeutics 17(4): 587-594.
Peters, U., J. Askling, et al. (2003). “Causes of death in patients with celiac disease in a population-based Swedish cohort.” Arch Intern Med 163(13): 1566-72.
Piper, J. L., G. M. Gray, et al. (2004). “Effect of prolyl endopeptidase on digestive-resistant gliadin peptides in vivo.” J Pharmacol Exp Ther 311(1): 213-9.
Pyle, G. G., B. Paaso, et al. (2005). “Effect of pretreatment of food gluten with prolyl endopeptidase on gluten-induced malabsorption in celiac sprue.” Clin Gastroenterol Hepatol 3(7): 687-94.
Quine, M. A., G. D. Bell, et al. (1995). “Prospective audit of upper gastrointestinal endoscopy in two regions of England: safety, staffing, and sedation methods.” Gut 36(3): 462-467.
Raivio, T. (2006). “Self transglutaminase-based rapid coeliac disease antibody detection by a lateral flow method.” Aliment Pharmacol Ther 24: 147-154.
Reunala, T. L. (2001). “Dermatitis herpetiformis.” Clinics in Dermatology 19(6): 728-736.
Rizzello, C. G., M. De Angelis, et al. (2007). “Highly efficient gluten degradation by lactobacilli and fungal proteases during food processing: new perspectives for celiac disease.” Appl Environ Microbiol 73(14): 4499-507.
Rostom, A., J. A. Murray, et al. (2006). “American Gastroenterological Association (AGA) Institute Technical Review on the Diagnosis and Management of Celiac Disease.” Gastroenterology 131(6): 1981-2002.
Rubio-tapia, et al. (2010) Am J Gastroenterol 105, 1412-1420.
Salmi, T. T. (2006). “Endomysial antibody-negative coeliac disease: clinical characteristics and intestinal autoantibody deposits.” Gut 55(1746-1753).
Salmi, T. T. (2006). “Immunoglobulin A autoantibodies against transglutaminase 2 in the small intestinal mucosa predict forthcoming coeliax disease.” Aliment Pharmacol Ther 24(541-552).
Sanderson (1975). “Failure of laboratory and radiological studies to predict jejunal mucosal atrophy.” Arch Dis Child 50: 526-31.
Shan, L., T. Marti, et al. (2004). “Comparative biochemical analysis of three bacterial prolyl endopeptidases: implications for coeliac sprue.” Biochem J 383(Pt 2): 311-8.
Shan, L., O. Molberg, et al. (2002). “Structural basis for gluten intolerance in celiac sprue.” Science 297(5590): 2275-9.
Shan, L., S. W. Qiao, et al. (2005). “Identification and analysis of multivalent proteolytically resistant peptides from gluten: implications for celiac sprue.” J Proteome Res 4(5): 1732-41.
Shiner (1973). “Ultrastructural changes suggestive of immune reactions in the jejunal mucosa of coeliac children following gluten challenge.” Gut 14 1-12
Shiner, M. and J. Ballard (1972). “Antigen-antibody reactions in jejunal mucosa in childhood coeliac disease after gluten challenge.” Lancet 1(7762): 1202-5.
Shiner, M. J. (1972). “Antigen-antibody reactions in jejunal mucosa in childhood coeliac disease after gluten challenge.” Lancet 1202-05.
Siegel, M., M. T. Bethune, et al. (2006). “Rational design of combination enzyme therapy for celiac sprue.” Chem Biol 13(6): 649-58.
Sollid, L. M. and C. Khosla (2005). “Future therapeutic options for celiac disease.” Nat Clin Pract Gastroenterol Hepatol 2(3): 140-7.
Stamnaes, J., B. Fleckenstein, et al. (2008). “The propensity for deamidation and transamidation of peptides by transglutaminase 2 is dependent on substrate affinity and reaction conditions.” Biochimica et Biophysica Acta (BBA)—Proteins & Proteomics 1784 (11): 1804-1811.
Stepniak, D., L. Spaenij-Dekking, et al. (2006). “Highly efficient gluten degradation with a newly identified prolyl endoprotease: implications for celiac disease.” Am J Physiol Gastrointest Liver Physiol 291(4): G621-9.
Stern, M. (2000). “Comparative Evaluation of Serologic Tests for Celiac Disease: A European Initiative Toward Standardization.” Journal of Pediatric Gastroenterology and Nutrition 31: 513-519.
Sturgess, R., P. Day, et al. (1994). “Wheat peptide challenge in coeliac disease.” The Lancet 343(8900): 758-761.
Sulkanen, S., T. Halttunen, et al. (1998). “Tissue transglutaminase autoantibody enzyme-linked immunosorbent assay in detecting celiac disease.” Gastroenterology 115(6): 1322-1328.
Svedlund, J., I. Sjodin, et al. (1988). “GSRS—a clinical rating scale for gastrointestinal symptoms in patients with irritable bowel syndrome and peptic ulcer disease.” Dig Dis Sci 33(2): 129-34.
Van Heel, D. A. and J. West (2006). “Recent advances in coeliac disease.” Gut 55(7): 1037-46.
Viljamaa, M., P. Collin, et al. (2005). “Is coeliac disease screening in risk groups justified? A fourteen-year follow-up with special focus on compliance and quality of life.” Alimentary Pharmacology & Therapeutics 22(4): 317-324.
Walker-Smith, J. (1990). “Revised criteria for diagnosis of coeliac disease.” Arch Dis Child 65: 909-11.
West, J. (2003). “Seroprevalence, correlates, and characteristics of undetected coeliac disease in England.” Gut 2003(52): 960-965.

We claim:

1. A method for reducing the severity and/or frequency of symptoms or clinical manifestations resulting from gluten ingestion, comprising administering to a seropositive patient with symptomatic celiac disease, gluten intolerance, or gluten sensitivity latiglutenase (ALV003) in an amount from about 100 mg to about 1200 mg per day.

2. The method of claim 1, wherein said patient has symptomatic celiac disease.

3. The method of claim 2, wherein said patient is moderately to severely symptomatic.

4. The method of claim 2, wherein the serology status of the patient is determined prior to ALV003 administration.

5. The method of claim 4, wherein determination of the serology status comprises an antibody test selected from the group consisting of anti-gliadin antibodies (AGA), anti-reticulin antibodies (ARA), IgA anti-human tissue transglutaminase (TTG) antibodies (TG2), IgA anti-endomysial antibodies (EMA), and anti-deamidated gliadin peptide (DGP) tests.

6. The method of any one of claims 1 to 5, wherein the patient is seropositive.

7. The method of any one of claims 1 to 5, wherein the patient remains seropositive despite adhering to a gluten-free diet.

8. The method of any one of claims 1 to 7, wherein the symptoms are self-reported.

9. The method of claim 8, wherein said patient has experienced moderately to severe symptoms of celiac disease within one month from first administration.

10. The method of any one of claims 1 to 9, wherein the symptoms of gluten ingestion are selected from the group consisting of abdominal pain, bloating, constipation and tiredness.

11. The method of claim 10, wherein said administration reduces the severity or frequency of at least one symptom selected from the group consisting of abdominal pain, bloating, constipation and tiredness.

12. The method of claim 11, wherein said administration reduces the severity or frequency of abdominal pain and bloating.

13. The method of claim 11, wherein said administration reduces the severity or frequency of constipation and tiredness.

14. The method of any one of claims 1 to 13, wherein said dose is 1200 mg of latiglutenase (ALV003).

15. The method of any one of claims 1 to 13, wherein said dose is 900 mg of latiglutenase (ALV003).

16. The method of any one of claims 1 to 13, wherein said dose is 600 mg of latiglutenase (ALV003).

17. The method of any one of claims 14 to 16, wherein said administering occurs at mealtime.

18. The method of claim 17, wherein said dose is administered with a major meal.

19. The method of claim 18, wherein said dose is administered with major meals three times per day.

20. The method of claim 18, wherein the daily amount is administered in one or more doses taken with food.

21. The method of claim 20, wherein said dose is administered at least once a day for at least a month.

22. The method of claim 20, wherein said dose is administered at least 300 days per year for at least two years.

23. The method of claim 20, wherein each dose comprises a dose of latiglutenase in powdered form and a dose of ALV002 and/or ALV001 in powdered form, and said powders are dissolved in a potable liquid to be ingested by said patient.

24. The method of any of claims 18 to 23, wherein said dose is administered with food containing at least 20 mg but not more than 20 g of gluten.

25. The method of claim 24, wherein said dose is administered with food containing no more than about 1 g of gluten.

26. The method of claim 25, wherein said dose is administered with food containing no more than about 5 g of gluten.

27. The method of claim 24, wherein said dose is administered with food containing no more than about 10 g of gluten.

28. An article of manufacture comprising a container with latiglutenase (ALV-003) therein, and a package insert, wherein the package insert provides instructions to administer latiglutenase for reducing the severity and/or frequency of symptoms or clinical manifestations resulting from gluten ingestion in a seropositive patient with symptomatic celiac disease, gluten intolerance, or gluten sensitivity.

29. The article of manufacture of claim 28, wherein the symptoms of gluten ingestion are selected from the group consisting of abdominal pain, bloating, constipation and tiredness.

30. The article of manufacture of claim 28 or 29, wherein said patient remains seropositive despite adhering to a gluten-free diet.

31. Use of latiglutenase (ALV003) in the preparation of a medicament for reducing the severity and/or frequency of symptoms or clinical manifestations resulting from gluten ingestion in a seropositive patient with symptomatic celiac disease, gluten intolerance, or gluten sensitivity.

32. The use of claim 31, wherein the symptoms of gluten ingestion are selected from the group consisting of abdominal pain, bloating, constipation and tiredness.

33. The use of claim 31 or 32, wherein said patient remains seropositive despite adhering to a gluten-free diet.

34. A medicament comprising latiglutenase (ALV003) for use in reducing the severity and/or frequency of symptoms or clinical manifestations resulting from gluten ingestion in a seropositive patient with symptomatic celiac disease, gluten intolerance, or gluten sensitivity.

35. The medicament of claim 34, wherein the symptoms of gluten ingestion are selected from the group consisting of abdominal pain, bloating, constipation and tiredness.

36. The medicament of claim 35, wherein said patient remains seropositive despite adhering to a gluten-free diet.
